(19) United States
(12) Patent Application Publication
(10) Pub. No.: US 2010/0248230 A1
(43) Pub. Date: Sep. 30, 2010

(54) NOVEL MEDICAL DIAGNOSTIC METHOD AND THERAPY IN THE CONTEXT OF INTERFREON-STIMULATED GENES THAT INDUCE DEPRESSION

(76) Inventors: Jörg Friedrich Schlaak, Essen (DE); Martin Trippler, Wuppertal (DE)

Correspondence Address: Brannon & Sowers PC, 1 North Pennsylvania Street, Suite 520, Indianapolis, IN, 46204, US

(21) Appl. No.: 12/595,747

(22) PCT Filed: Apr. 4, 2008
(86) PCT No.: PCT/EP08/02765

§ 371 Date: Oct. 13, 2009

(30) Foreign Application Priority Data
Apr. 14, 2007 (DE) 10 2007 017 986.5
Apr. 16, 2007 (DE) 10 2007 018 136.3
May 14, 2007 (DE) 10 2007 022 550.6

(51) Int. Cl. C12Q 1/68

(52) U.S. Cl. 435/6

(57) ABSTRACT

The invention relates to a novel medical diagnostic method and treatment in conjunction with depression-inducing genes which are stimulated in particular by interferon. The invention relates in particular to the use of at least one nucleic acid molecule which induces depression and/or is associated with depression, in particular a gene and/or the DNA sequence thereof and/or the RNA sequence associated therewith, and/or a (poly)peptide encoded by the nucleic acid, for finding and/or providing a diagnostic method for detecting depression, or a medicament for the preventive and/or curative treatment of depression, and/or for determining the risk of developing depression, and/or for predicting the individual effects and/or side-effects of medicaments, in particular for treatment with interferon (for example, for the treatment of hepatitis).

### CROSS-REFERENCES TO RELATED APPLICATIONS

This application is a National Stage filing of International Application PCT/EP2008/002765, filed Apr. 4, 2008, claiming priority to German Application Nos. DE 10 2007 017 986.5 filed Apr. 14, 2007, DE 10 2007 018 136.3, filed Apr. 16, 2007, and DE 10 2007 022 550.6 filed May 14, 2007. The subject application claims priority to PCT/EP2008/002765 and to German Application Nos. DE 10 2007 017 986.5, DE 10 2007 018 136.3, and DE 10 2007 022 550.6 and incorporates all by reference herein, in their entirety.

### BACKGROUND OF THE INVENTION

The present invention relates to the identification of genes which are involved in the development of depression, in particular depression induced by interferon, and/or endogenous depression. In this respect the present invention likewise relates to a method for identifying said genes, whereby the levels or rates of expression of the genes which are involved in interferon-induced depression and/or endogenous depression are determined and correlated before and after administration of interferon, in particular α-interferon, or in test subjects with or without endogenous depression. The genes determined in this manner, which are involved in interferon-induced depression, and are also referred to in particular as interferon-stimulated depression-inducing genes, and/or genes involved in endogenous depression, are DYNLT1, MEF2A, TOR1B, or DISC1, for example.

The present invention further relates to the use of a nucleic acid molecule which induces depression or is associated with depression, in particular a gene which induces depression or is associated with depression, for diagnosis and treatment, for predicting disease progressions, and/or for predicting the side effects or the response to medicaments.

The present invention further relates to methods for identifying substances which regulate the gene activity of the genes or corresponding gene products which induce depression or are associated with depression, and a method for improving the pharmacological properties of these substances. The present invention further relates to the use of substances that regulate the gene activity of a gene which induces depression or is associated with depression in the area of diagnosis and treatment of depression.

Lastly, the present invention relates to a method for determining the risk of a test subject of developing depression.

Within the scope of the present invention, depression is preferably considered to be depression that occurs as an undesired side effect of interferon therapy within the scope of interferon-assisted treatment of hepatitis, in particular type C hepatitis.

With approximately 170 million cases worldwide, chronic infection with the hepatitis C virus (HCV) is a significant global health problem. With a chronification rate of approximately 80%, type C hepatitis represents one the primary causes of hepatitis, cirrhosis of the liver, and liver cell carcinoma. As the most efficient treatment for chronic type C hepatitis, interferon (IFN), in particular α-interferon (also referred to as interferon-alpha or IFNα), preferably pegylated IFNα, optionally in combination with ribavirin, is used in the prior art. One of the most common side effects of this treatment is IFN-induced severe depression, which in addition to impairing quality of life may also result in discontinuation of treatment, or even suicide.

Various studies have shown that in the treatment of type C hepatitis (also referred to as hepatitis C), in particular chronic hepatitis C, with pegylated α-interferon, the development of depression in varying levels of severity was statistically significantly higher in test subjects. Additional studies have even shown that symptoms of depression also had direct negative effects on the therapy in terms of reduction of the viral load, and as such therefore adversely affected the treatment of type C hepatitis. In addition, other studies have shown that in comparison to other side effects for the test subjects or patients, the incidence of depression represents the greatest burden resulting from the treatment of type C hepatitis with interferon. Further information on this subject may be obtained from the publication by Younossi, Z. et al., “The effects of HCV infection and management on health-related quality of life,” Hepatology, 2007, Vol. 45, pages 806-816, and the literature cited therein.

Similarly, within the scope of the present invention endogenous depression may be involved, as described above.

Depression, also referred to as depressive episode or recidivistic depressive disorder, is a psychological condition requiring treatment, and is often characterized by the combined appearance of symptoms such as reduced drive and mental process, flattened mood, restlessness, and sleep disturbances. The aforementioned flattened mood is often accompanied by increased irritability and anxiety, often with excessive emphasis on negative thoughts and failure to perceive positive aspects, or ascribing such to coincidence. Depressive disorders are frequently associated with physical symptoms such as loss of appetite, weight loss, weight gain, and pain in various areas of the body, and often heightened susceptibility to infection during a depressive episode. Depending on the severity, depression may be accompanied by latent or acute suicidal ideation. It is suspected that the majority of the approximately 12,000 suicides per year in Germany are attributable to depression. Social withdrawal is often associated with depression.

In the prior art, the agent of choice for treatment of depression is frequently pharmacotherapy involving the use of antidepressant medications.

These include, for example, selective serotonin reuptake inhibitors (SSRI), which are based on the activity mechanism of relative inhibition of the selective reuptake of serotonin at the presynaptic membrane, resulting in a relative increase in the transmitter serotonin. However, sexual dysfunction and anorgasmia are frequent side effects. In addition, these substances sometimes cause an initial increase in motivation, followed only later by mood elevation, which may result in higher risk of suicide in the first weeks of taking the medication. In the United States, these types of medications have recently been required to display an appropriate warning advisory.

Tricyclic antidepressants are also used. The primary disadvantage lies in their side effects, such as dry mouth, constipation, fatigue, muscle tremors, and decreased blood pressure. Furthermore, tricyclic antidepressants as well cause an initial increase in motivation, followed only later by mood elevation.

Monoaminooxidase inhibitors (MAO inhibitors), which block the enzyme monoaminooxidase which splits amines such as serotonin and noradrenalin, represent an additional substance class for the treatment of depression. However, patients treated with this type of therapeutic agent must maintain a strict diet low in tyramine in order to avoid a dangerous rise in blood pressure.

Disadvantages of the above-referenced substances are their severe side effects, and the fact that therapeutic success is not always guaranteed.

In contrast, the underlying activity mechanism of interferon-induced depression is not understood. There are various hypotheses which implicate the influence of interferon on glucocorticoid or serotonin-1A (5-HT) receptors. It is further assumed that the administration of interferon for treatment of type C hepatitis results in increased concentrations of adrenocorticotrophic hormone (ACTH), cortisol, and interleukin-6 in patients who develop depression during treatment with interferon. On this basis, also for treatment of depression occurring in conjunction with hepatitis C treated by interferon, the targeted administration of selective serotonin reuptake inhibitors (SSRI) is proposed in the prior art. For further information and reports on this subject, reference is made to the publication by Horsmans, Y., “Interferon-induced depression in chronic hepatitis C,” Journal of Antimicrobial Chemotherapy, 2006, Vol. 58, pages 711-713, and the literature cited therein.

With regard to a possible genetic predisposition toward development of depression, the prior art provides an incomplete and inconsistent explanation, whereby it can only be stated with certainty that there is no such thing as a single, isolated “depression gene” in a manner of speaking; rather, regulation of a number of genes in particular may result in depression.

Thus, the prior art heretofore has provided no explanation at all concerning the extent to which a specific genetic predisposition is involved in the development of endogenous as well as interferon-induced depression. In particular, no specific gene has been discovered thus far which influences or causes the development of depression, especially interferon-induced depression and endogenous depression. This is because specific genes which participate or play a crucial role in the development of interferon-induced depression, in particular in relation to the treatment of type C hepatitis, or also endogenous depression, have not yet been identified.

However, the identification of such genes would represent a major step forward in an effective therapy or treatment of depression, in particular the type of depression associated with the therapeutic administration of interferon for the treatment of type C hepatitis. In particular for patients already suffering from type C hepatitis, development of depression represents an enormous additional burden and influence on the quality of life, especially since the patients may already be significantly weakened as the result of pre-existing illness.

US 2001/0029015 A1 relates to a method for detecting mutations and polymorphisms in the torsin gene, torsin-related genes, and methods for detecting neuronal diseases mediated by these mutations and polymorphisms. The cited document concerns the diagnosis of neuronal diseases by use of single-nucleotide polymorphism (SNP)-based analyses.

Furthermore, US 2003/0054345 A 1 relates to a method and a composition for diagnosing and treating neuropsychiatric disorders such as schizophrenia. In this regard the gene DISC1 is referenced, and the above-described conditions are discussed on the basis of molecular-biological analyses of DISC1.

Lastly, US 2005/0255500 A1 also relates to psychiatric disorders associated with the DISC1 gene, and addresses the distribution of DISC1 in bodily cells.

The above-referenced documents of the prior art are not directed to the targeted treatment of depression, in particular with regard to optimization of the treatment of interferon-induced depression, in particular for treatment of type C hepatitis, or the treatment of endogenous depression.

In light of the above, it is an object of the present invention to provide novel and efficient diagnostic and treatment options for endogenous and interferon-induced depression, in particular depression associated with excessive activity of certain genes, with the aim of greater efficacy and at the same time reduced side effects compared to approaches known from the prior art.

### BRIEF SUMMARY OF THE INVENTION

In this respect, a further object of the present invention is to provide a method by which specific genes may be identified which have crucial involvement in the development of depression, in particular endogenous as well as interferon-induced depression, for example in the treatment of type C hepatitis.

A further object of the present invention is to provide a method for identifying substances, or substances as such, by means of which the gene expression or gene activity of genes which induce depression or are associated with depression may be controlled in a targeted manner.

Lastly, it is an object of the invention to provide methods and uses of the above-referenced type which allow the disadvantages of the prior art to be avoided or at least reduced.

In other words, it is an object of the invention to identify relevant risk factors of interferon-induced and/or endogenous depression, and to use same for the development of preventative treatment strategies.

### BRIEF DESCRIPTION OF THE SEVERAL VIEWS OF THE DRAWINGS

Abbreviations have the following meanings in the drawings: n or N=normal, D=depressive, HCV=hepatitis C virus infection, SDE=severe depressive episode, n.s.=not significant, Ko=control, HCV/D=hepatitis C virus infection, depression.

FIG. 1 provides the results of GeneChip analyses for the expression of genes which induce depression and/or are associated with depression in type C hepatitis patients undergoing Pegasys treatment.

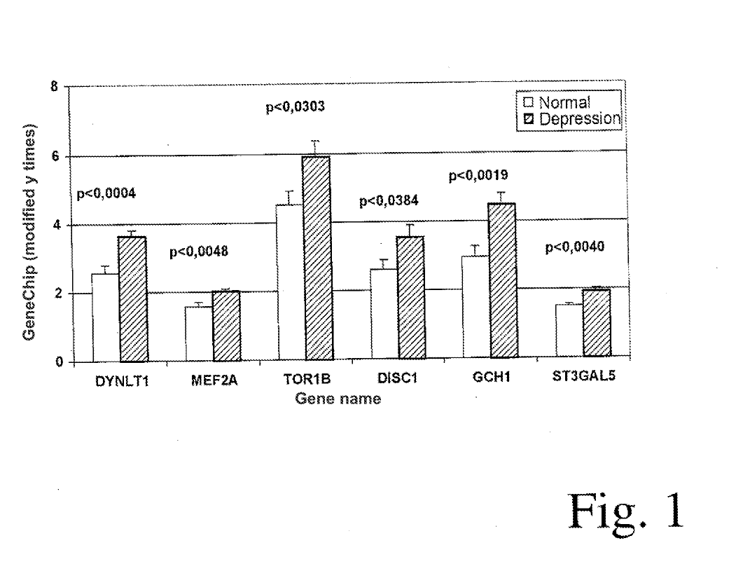

FIG. 2 provides the results of GeneChip analyses for the expression of genes which induce depression and/or are associated with depression in patients with type C hepatitis, with and without interferon-induced depression.

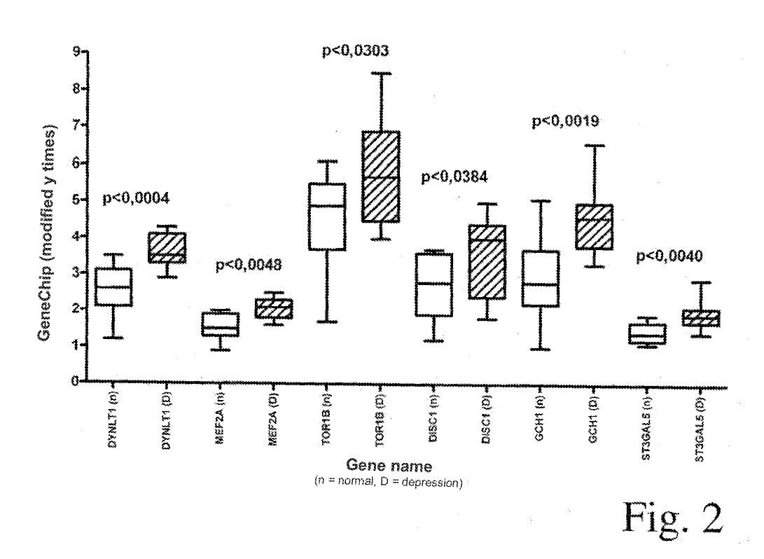

FIG. 3 provides GeneChip data for the expression of further genes which induce depression and/or are associated with depression in type C hepatitis patients undergoing Pegasys treatment.

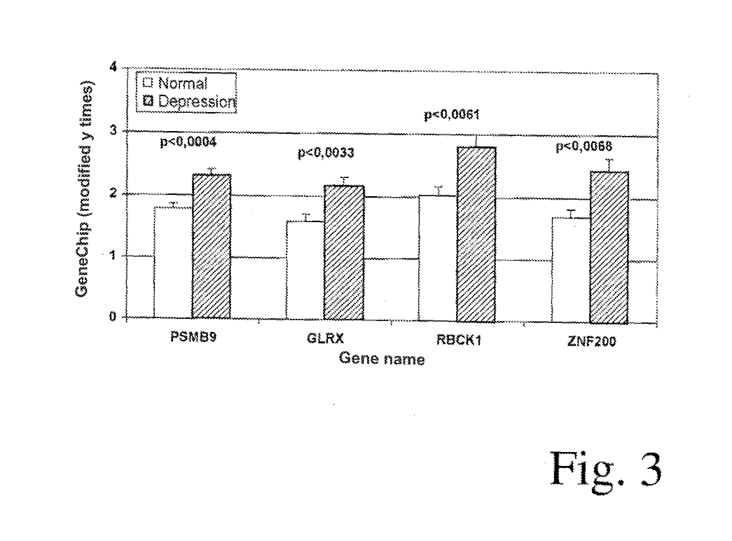

FIG. 4 provides GeneChip data for the expression of further genes which induce depression and/or are associated with depression in type C hepatitis patients, with and without IFN-induced depression.

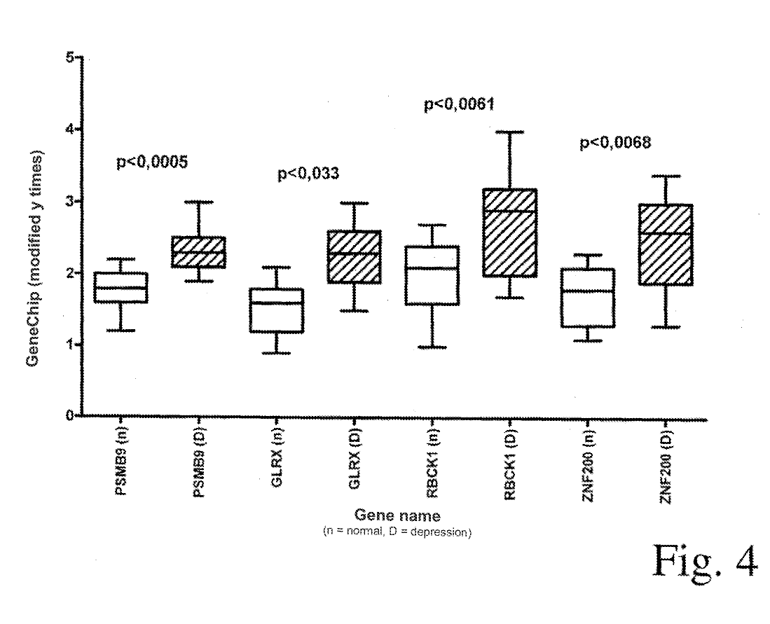

FIG. 5 provides the results of GeneChip analyses for the expression of IFN response genes whose expression is correlated with IFN-induced depression in type C hepatitis patients undergoing Pegasys treatment.

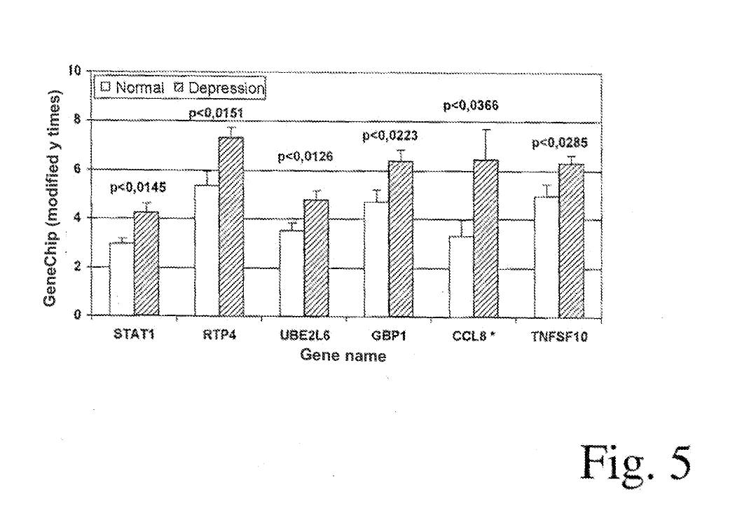

FIG. 6 provides the results of GeneChip analyses for the expression of IFN response genes whose expression is correlated with IFN-induced depression in type C hepatitis patients, with and without IFN-induced depression.

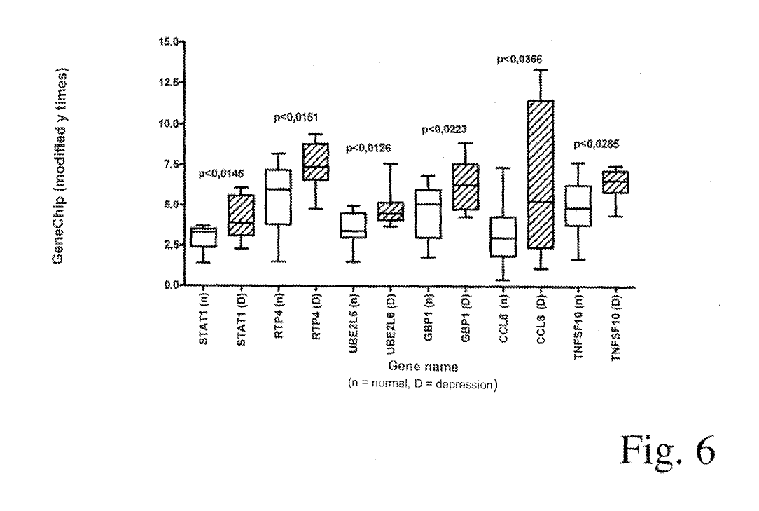

FIG. 7A provides the gene expression of GCH1 and TOR1B in patients with hepatitis C virus (HCV) and psychiatric patients with a severe depressive episode (SDE), and a comparison with control groups.

FIG. 7B provides the gene expression of DYNLT1 and DISC1 in patients with HCV and psychiatric patients with an SDE, and a comparison with control groups.

FIG. 7C provides the gene expression of MEF2A and ST3GAL5 in patients with HCV and psychiatric patients with an SDE, and a comparison with control groups.

FIG. 7D provides the gene expression of MX1 and ISG15 in patients with HCV and psychiatric patients with an SDE, and a comparison with control groups.

FIG. 8 A provides the gene expression of STAT1, IFIT1, ISG15, and MX1 in psychiatric patients with an SDE.

FIG. 8B provides the IFN production for various patient groups.

### DETAILED DESCRIPTION OF THE INVENTION

Within the scope of the present invention, the applicant has surprisingly been able for the first time to identify genes which are involved in the development of depression, in particular interferon-induced depression, as well as endogenous depression. In this regard the applicant has surprisingly found that development of depression is associated with an increase in the specific gene activity of defined genes. The genes identified within the scope of the present invention have not previously been linked to the development of interferon-induced or interferon-mediated depression, and are used as the basis for early detection of interferon-induced depression and for preparing new medicaments for the treatment of interferon-induced depression. According to the invention, however, the identified genes may also be used as the basis for methods or medicaments of the aforementioned type for the treatment of endogenous depression, or as the basis for early detection of endogenous depression. Also with regard to endogenous depression, as previously stated the applicant has surprisingly found that an increase in gene activity of certain genes is present. In addition, with regard to endogenous depression the applicant has observed upregulation in the production of interferons, which in turn may contribute to activation of certain genes.

According to a first aspect of the present invention, the invention therefore relates to the use according to claim 1, i.e., use of at least one nucleic acid molecule which induces depression and/or is associated with depression, in particular a gene and/or the DNA sequence thereof and/or the associated RNA sequence thereof, and/or at least one (poly)peptide encoded by the nucleic acid, for finding and/or providing a diagnostic method for the detection of depression, and/or a medicament for the preventive and/or curative treatment of depression, and/or for determining the risk of developing depression, and/or for predicting the individual effects and/or side-effects of medicaments (in particular for treatment with interferon, for example for the treatment of hepatitis).

In other words, the applicant has surprisingly found that specific genes are involved in the development of depression, in particular interferon-induced depression. The genes are in particular the following:
DYNLT1, in particular having the transcript ID (locus) NM_{—}006519, in particular according to SEQUENCE LISTING SEQ. ID. NO. 1 and/or Table 1,
MEF2A, in particular having the transcript ID (locus) NM_{—}005587, in particular according to SEQUENCE LISTING SEQ. ID. NO. 2 and/or Table 1,
TOR1B, in particular having the transcript ID (locus) NM_{—}014506, in particular according to SEQUENCE LISTING SEQ. ID. NO. 3 and/or Table 1,
DISC1, in particular having the transcript ID (locus) NM_{—}018662, in particular according to SEQUENCE LISTING SEQ. ID. NO. 4 and/or Table 1,
GCH1, in particular having the transcript ID (locus) NM_{—}000161, in particular according to SEQUENCE LISTING SEQ. ID. NO. 5 and/or Table 1,
ST3GAL5, in particular having the transcript ID (locus) NM_{—}003896, in particular according to SEQUENCE LISTING SEQ. ID. NO. 6 and/or Table 1,
PSMB9, in particular having the transcript ID (locus) NM_{—}002800, in particular according to SEQUENCE LISTING SEQ. ID. NO. 7 and/or Table I,
GLRX, in particular having the transcript ID (locus) NM_{—}002064, in particular according to SEQUENCE LISTING SEQ. ID. NO. 8 and/or Table 1,
RBCK1, in particular having the transcript ID (locus) NM_{—}006462, in particular according to SEQUENCE LISTING SEQ. ID. NO. 9 and/or Table 1, and
ZNF200, in particular having the transcript ID (locus) NM_{—}003454, in particular according to SEQUENCE LISTING SEQ. ID. NO. 10 and/or Table 1,
STAT1, in particular having the transcript ID (locus) NM_{—}007315, in particular according to SEQUENCE LISTING SEQ. ID. NO. 11 and/or Table 2,
RTP4, in particular having the transcript ID (locus) NM_{—}022147, in particular according to SEQUENCE LISTING SEQ. ID. NO. 12 and/or Table 2,
UBE2L6, in particular having the transcript ID (locus) NM_{—}004223, in particular according to SEQUENCE LISTING SEQ. ID. NO. 13 and/or Table 2,
GBP1, in particular having the transcript ID (locus) NM_{—}002053, in particular according to SEQUENCE LISTING SEQ. ID. NO. 14 and/or Table 2,
CCL8, in particular having the transcript ID (locus) NM_{—}005623, in particular according to SEQUENCE LISTING SEQ. ID. NO. 15 and/or Table 2,
TNFSF 10, in particular having the transcript ID (locus) NM_{—}003810, in particular according to SEQUENCE LISTING SEQ. ID. NO. 16 and/or Table 2,
   or combinations thereof.

As discussed below in greater detail, the above-referenced genes are genes which induce depression and/or are associated with depression, and/or IFN response genes whose expression is correlated with IFN-induced expression.

The sequence identification numbers 1 through 16 (SEQ. ID. NO. 1 through SEQ. ID. NO. 16), which are listed above as SEQUENCE LISTING and are summarized in the appendix for the sequence protocols, refer to the respective DNA sequences for the above-referenced genes. Sequence identification numbers 1 through 16 according to the sequence protocols were created on the basis of data or information standardized under patent law, using PatentIn Version 3.3 software.

Of the genes listed above, preferred according to the invention are those from the group comprising DYNLT1, MEF2A, TOR1B, DISC1, GCH1, ST3GAL5, PSMB9, GLRX, RBCK1, and ZNF200, in particular as defined above. For the above-referenced genes the applicant was able to determine a high degree of influence on the development of depression, as illustrated in the exemplary embodiments.

Particularly preferred according to the invention are genes from the group comprising DYNLT1, MEF2A, TOR1B, DISC1, GCH1, and ST3GAL5, in particular as defined above. For the above-referenced genes the applicant was able to determine a particularly high degree of influence on the development of depression, as illustrated in the exemplary embodiments.

With regard to the underlying depression, a depressive condition associated with the treatment or therapy of hepatitis, in particular chronic type C hepatitis, is involved which, within the scope of the present invention, includes all levels or degrees of severity of depression. Stated more precisely, the depression may involve a condition which is associated with or caused by α-interferon, in particular pegylated α-interferon, in particular in which the depression is caused by the preferably systemic administration of α-interferon, in particular pegylated α-interferon, especially in the treatment or therapy of hepatitis, in particular chronic type C hepatitis.

In other words, the depression under discussion is a condition in particular which is associated with or caused by increased gene activity or gene expression of at least one of the above-referenced genes. In this regard, the gene activation may be associated with or caused by, for example, the preferably systemic administration of α-interferon, in particular pegylated α-interferon, especially in the treatment or therapy of hepatitis, in particular chronic type C hepatitis.

In addition, the depression may be endogenous depression, which similarly may be associated with or caused by increased gene activity or gene expression of at least one of the above-referenced genes.

The term “gene” used according to the invention likewise includes the corresponding nucleic acid molecule or the corresponding DNA sequence. However, the use according to the invention likewise refers to the RNA sequence associated with the gene which, so to speak, may function as a post-transcriptional product which is complementary to the codogenic strand of DNA of the gene. Similarly, this term may also be used within the scope of the invention to refer to the (poly)peptide, and thus in a manner of speaking, the translation product, encoded by the nucleic acid or the gene, in particular as defined above. The term “nucleic acid molecule” is synonymous with the term “polynucleic acid” or “polynucleic acid molecule.”

With regard to the use according to the invention, the gene which induces depression and/or is associated with depression may be used, in a manner of speaking, as an initial object for finding or providing diagnostic methods or medicaments for depression of the type described above. The diagnostic methods or medicaments may be such that they interact directly or indirectly with the gene, or the RNA and/or the corresponding (poly)peptide, which induces depression and/or is associated with depression, in order to prevent the development of depression or at least lessen its severity. These may be substances which have a gene-regulating effect. In addition, the above-referenced compounds which induce depression and/or are associated with depression, in particular the genes referenced above, may be used for providing a diagnostic method, whereby substances may be provided which, for example, allow a diagnosis of depression on the basis of interaction with the genes which induce depression and/or are associated with depression, and, for example, for increased gene activity of the genes which induce depression and/or are associated with depression, a conclusion may be drawn concerning the presence of depression.

With regard to the use of compounds which induce depression and/or are associated with depression for determining the risk of developing depression, the above-referenced substances may be used, for example, in statistical analysis and evaluation methods in order to assign an increased risk of a patient's developing depression, for example within the scope of interferon therapy, provided that, for example, increased gene activity of the gene which induces depression and/or is associated with depression is present. It is likewise possible by the use according to the invention to predict individual effects of medicaments, for example interferon, in particular α-interferon, preferably pegylated α-interferon, especially with regard to development of depression, as well as effects of antidepressants and the like, in particular with regard to avoidance of depression, or to estimate the side effects thereof.

The principles concerning the specific fields of application of the use according to the invention are largely known to one skilled in the art, and therefore do not require further discussion.

According to a second aspect of the present invention, the invention relates to a method according to claim 2, i.e., a method for identifying an inhibitor and/or repressor of a nucleic acid molecule which induces depression and/or is associated with depression, in particular a gene and/or the DNA sequence thereof and/or the RNA sequence associated therewith, comprising the following steps:
(a) Bringing the nucleic acid molecule into contact with at least one test substance under conditions that allow interaction, in particular binding, of the test substance(s) to the nucleic acid molecule; and
(b) Detecting and/or analyzing whether the test substance(s) limit or prevent the gene activity and/or expression of the nucleic acid molecule, and/or whether the test substance(s) limit or prevent the depression-inducing and/or depression-associated properties of the nucleic acid molecule.

The method according to the invention may be carried out in vitro, for example, whereby in a manner of speaking an interaction of the test substance with the nucleic acid molecule or the gene may be investigated, and in the presence of an interaction a conclusion may be drawn concerning decreased gene activity as a result of this interaction. The method according to the invention may likewise be carried out in a corresponding host system, whereby the host is a carrier of the nucleic acid molecule or gene which induces depression and/or is associated with depression, and advantageously has a corresponding expression system. The method according to the invention may likewise be used for identification of IFN response genes. The interaction or the influence of gene activity may be detected using methods known to one skilled in the art.

In this regard, according to a third aspect of the present invention, the invention relates to a method according to claim 3, i.e., a method for identifying an inhibitor and/or repressor of a (poly)peptide that is encoded by a nucleic acid molecule which induces depression and/or is associated with depression, in particular a gene and/or the DNA sequence thereof and/or the associated RNA sequence thereof, comprising the following steps:
(a) Bringing the (poly)peptide into contact with at least one test substance under conditions that allow interaction, in particular binding, of the test substance(s) to the (poly)peptide;
(b) Detecting and/or analyzing whether the test substance(s) limit or prevent the depression-inducing and/or depression-associated properties of the (poly)peptide.

Thus, according to this aspect the method according to the invention focuses on influencing the gene product. The method may be carried out, in a manner known to one skilled in the art, in vitro as well as in vivo in a host system, whereby in the latter case the host should preferably bear the nucleic acid that encodes for the (poly)peptide to be investigated. Similarly, an interaction may also be achieved in vitro on isolated (poly)peptides. The method according to the invention may likewise be used with regard to IFN response genes.

The methods according to the invention corresponding to the second and third aspects of the present invention may be carried out in such a way that multiple test substances are used and the following steps are carried out:
(a) Testing various test substances in different reaction vessels, whereby test substances which do not limit or prevent the depression-inducing and/or depression-associated properties of the nucleic acid molecule and/or the (poly)peptide are not taken into account in the subsequent test method;
(b) Distributing test substances from such reaction vessels, in which reduction or prevention of depression-inducing and/or depression-associated properties of the nucleic acid molecule and/or (poly)peptide was determined in step (a), into new reaction vessels and repeating step (a) with the new reaction vessels; and
(c) Repeating step (b) until a single test substance is identified which may be associated with reduction or prevention of depression-inducing and/or depression-associated properties of the nucleic acid molecule and/or (poly)peptide.

Similarly, the method may be carried out according to the second and third aspects of the present invention in such a way that the test substance(s), the nucleic acid molecule, and/or the (poly)peptide are linked to a readout system, and/or wherein the test assay is added to a readout system, and/or wherein the readout system sends a detectable signal after the test substance(s) bind to the nucleic acid molecule and/or the (poly)peptide.

Within the scope of the above-referenced method, the test substances may be low-molecular substances, peptides, aptamers, antibodies, and/or fragments or derivatives thereof.

As previously stated, the above-referenced methods may be carried out in a host or host system, for example, which preferably includes the genes defined above and also has a corresponding expression system. One skilled in the art is familiar with such hosts, or is able at any time to select specific host systems on the basis of the present invention; therefore no further explanation is needed. The methods according to the invention may likewise be carried out in the form of high-throughput processes and/or with computer assistance.

For further particulars regarding the methods according to the invention corresponding to the second and third aspects of the present invention, reference is made to the discussion of the first two referenced aspects or subject matter of the present invention, which correspondingly apply.

According to a fourth aspect of the present invention, the invention relates to a method according to claim 10, i.e., a method for improving the pharmacological properties of the test substances identified corresponding to the third aspect of the present invention according to the previously described method, wherein
(a) the binding site of the test substance to the nucleic acid molecule or to the (poly)peptide, and optionally the binding site of the nucleic acid molecule or the (poly)peptide to the test substance, is identified;
(b) the binding site of the test substance and of the nucleic acid molecule or of the (poly)peptide is modified by molecular modeling; and
(c) the test substance is modified in such a way that its binding specificity or binding affinity or binding avidity for the nucleic acid molecule or the poly)peptide is increased.

In this regard, the binding site in step (a) may be determined by location-specific mutagenesis, the applicable methods being known as such to one skilled in the art.

According to a fifth aspect of the present invention, the invention further relates to a method according to claim 12, i.e., a method for modifying a test substance which is identified or improved according to the previously defined method, wherein the test substance is further modified as a lead structure to achieve
(i) a modified active center, a modified activity spectrum, and/or a modified organ specificity; and/or
(ii) improved activity; and/or
(iii) reduced toxicity (improved therapeutic index); and/or
(iv) reduced side effects; and/or
(v) time-shifted onset of the therapeutic activity and/or duration of the therapeutic activity; and/or
(vi) altered pharmacokinetic parameters (in particular absorption, distribution, metabolism, and/or excretion); and/or
(vii) modified physicochemical parameters, in particular solubility, hygroscopic properties, color, taste, odor, stability, and/or physical state; and/or
(viii) improved general specificity or organ/tissue specificity; and/or
(ix) an optimized administration form and/or route, in particular by
   (a) esterification of carboxyl groups and/or
   (b) esterification of hydroxyl groups with carboxylic acids and/or
   (c) esterification of hydroxyl groups, in particular to produce phosphates, pyrophosphates, or sulfates and/or hemisuccinates and/or
   (d) formation of pharmaceutically acceptable salts and/or
   (e) formation of pharmaceutically acceptable complexes and/or
   (f) synthesis of pharmacologically active polymers and/or
   (g) introduction of hydrophilic groups and/or
   (h) introduction and/or substitution of substituents in aromatic compounds and/or side chains, and/or alteration of the substituent pattern and/or
   (i) modification by introduction of isosteric and/or bioisosteric groups and/or
   (j) synthesis of homologous compounds and/or
   (k) introduction of branched side chains and/or
   (l) conversion of alkyl substituents to cyclic analogs and/or
   (m) derivatization of hydroxyl groups to produce ketals and/or acetals and/or
   (n) N-acetylation to produce amides and/or phenylcarbamates and/or
   (o) synthesis of Mannich bases and/or imines and/or
   (p) conversion of ketones and/or aldehydes to Schiff bases, oximes, acetals, ketals, enol esters, oxazolidines, thiozolidines, or combinations thereof.

Within the scope of the method according to the invention it is possible to achieve further pharmaceutical improvement of the identified, improved, or modified test substance, in particular the inhibitor or repressor of the above-referenced genes, by use of peptidomimetics.

For further discussion regarding the methods according to the invention corresponding to the fourth and fifth aspects of the present invention, reference is made to the discussion of the aforementioned aspects of the present invention, which correspondingly apply.

According to a sixth aspect of the present invention, the invention further relates to a method according to claim 14, i.e., use of an inhibitor and/or repressor of a nucleic acid molecule which induces depression and/or is associated with depression, in particular a gene and/or the DNA sequence thereof and/or the RNA sequence associated therewith, for producing a medicament for the preventative and/or curative treatment of depression. However, the gene may also be an IFN response gene.

The genes employed within the scope of the use according to the invention are preferably the genes defined above. In this regard the applicant has surprisingly found that the genes identified thereby may be selected as the basis for a therapeutic approach in order to greatly reduce or prevent the development of depression such as endogenous depression, and depression in particular in patients treated with interferon, in particular α-interferon, for treatment of type C hepatitis, for example.

In addition, the development or onset of depression may be prevented in a very early stage by early administration of the inhibitors or repressors in the course of preventative treatment. Thus, targeted pharmacological suppression of the activity of the genes which induce depression and/or are associated with depression, in particular as defined above, results in a targeted treatment of depression. The inhibitors or repressors employed within the scope of the use according to the invention may in particular be a test substance identified according to the above-referenced method according to the invention, which in a manner of speaking acts as an inhibitor or repressor with regard to the corresponding target gene, in particular as defined above.

Furthermore, the (poly)peptide encoded by the gene which induces depression and/or is associated with depression may also be regarded as a target for a therapeutic approach.

According to a seventh aspect of the present invention, the invention relates to a method according to claim 15, i.e., the use of an inhibitor and/or repressor of a (poly)peptide that is encoded by a nucleic acid molecule which induces depression and/or is associated with depression, in particular a gene and/or the DNA sequence thereof and/or the RNA sequence associated therewith, for producing a medicament or medicinal product for the preventative and/or curative treatment of depression.

A test substance identified as an inhibitor or repressor may also be used in this regard in the method described above.

According to an eighth aspect of the present invention, the invention relates to a method according to claim 17, i.e., the use according to the invention of at least one substance for producing a medicament or drug for the preventative and/or curative treatment of depression, whereby the substance regulates, in particular reduces or at least inhibits, the gene activity and/or gene expression of at least one gene which induces depression and/or induces [sic; is associated with] depression.

In this manner depression may be suppressed in a targeted manner by the targeted setting or regulation or reduction of the gene activity. Thus, as surprisingly found by the applicant, the development of depression, in particular interferon-induced depression as well as endogenous depression, is associated with increased gene activity of the gene which induces depression and/or is associated with depression.

According to one embodiment preferred according to the invention, the substance is able to interact with the promoter and/or enhancer of the gene which induces depression and/or is associated with depression in such a way that the binding in particular of endogenous transcription factors, in particular activators, to the promoter and/or enhancer is prevented or at least inhibited.

Likewise, it is also possible for the substance to interact in particular with an endogenous transcription factor, in particular an activator, in such a way that the binding of the transcription factor, in particular the activator, to the promoter and/or enhancer of the gene which induces depression and/or induces [sic; is associated with] depression is prevented or at least inhibited.

Similarly, it is also possible for the substance to react with the endogenous transcription activators themselves, thus causing inactivation of the transcription activator as such, in order to reduce the gene activity.

The substance employed within the scope of the use according to the invention may be a substance identified using the method according to the invention described above.

For further particulars regarding the use according to the invention corresponding to the sixth, seventh, and eighth aspects of the present invention, reference is made to the discussion of the other aforementioned aspects of the present invention, which correspondingly apply.

According to a ninth aspect of the present invention, the invention relates to a method according to claim 20, i.e., a method for determining the risk of a test subject of developing depression and/or for predicting individual effects and/or side effects of medicaments, in particular depression as a result of interferon therapy, wherein an elevated risk of developing [depression] and/or altered drug effects and/or side effects are assigned to the test subject for the case that the test subject has increased gene activity and/or gene expression of at least one gene which induces depression and/or is associated with depression.

According to this subject matter, the method according to the invention may be carried out using an expression profile, for example, in particular within the scope of interferon therapy, whereby the expression profile may be balanced, for example with respect to a control group that has not been treated with interferon, or with respect to a control group treated with interferon whose subjects have not developed depression, and an elevated risk of developing [depression] is assigned to the affected test subjects when increased gene activity is detected with regard to the previously defined genes which induce depression and/or are associated with depression, for example. With regard to information concerning altered effects or side effects of medicaments, this may, for example, relate specifically to the interferon used in the treatment of type C hepatitis. Thus, for example, an increased side effect may be assigned to the pharmacological substance, especially interferon, in particular with respect to development of depression, with regard to the respective test subjects when increased gene activity is detected for the genes in question which induce depression and/or are associated with depression.

Accordingly, corresponding to this aspect of the present invention within the scope of the method according to the invention, the medicament in particular involves α-interferon, in particular pegylated α-interferon, administered in the treatment or therapy of hepatitis, in particular chronic type C hepatitis, whose side effects may be determined with regard to the development of depression. This allows better assessment of a therapeutic approach or course of treatment in the administration of interferon for type C hepatitis.

Lastly, according to a tenth aspect of the present invention, the invention relates to a method according to Claim 22, i.e., a method for identifying and/or determining at least one nucleic acid molecule which induces depression and/or is associated with depression, in particular a gene, preferably a gene which induces depression and/or is associated with depression in conjunction with the administration of interferon, wherein the method comprises the following steps:
(a) Creation of a gene expression and/or gene activity profile for a number of test subjects of a test group treated with interferon;
(b) Analysis and comparison or balancing of the respective gene expression and/or gene activity profiles of (i) test subjects who develop depression as the result of interferon therapy and (ii) test subjects who do not develop depression as the result of interferon therapy; and
(c) Identification of at least one nucleic acid molecule, in particular at least one gene, having increased gene expression and/or gene activity in (i) test subjects with depression compared to (ii) test subjects without depression.

The method may include the following step after step (c):
(d) Assignment of the nucleic acid molecule, in particular the gene, identified in step (c) as a nucleic acid molecule which induces depression and/or is associated with depression, in particular a gene which induces depression and/or is associated with depression, preferably as a gene which induces depression and/or is associated with depression in conjunction with the administration of interferon.

According to this aspect of the present invention, the method according to the invention may be used, for example, on the basis of differential expression, employing so-called DNA chips. The procedure may be carried out, for example, in such a way that first RNA is isolated from the blood of a test subject to be examined, and this isolate is provided on specialized gene chips, and by use of evaluation and analytical methods known as such to one skilled in the art the rate of expression for certain genes is determined, and the characteristics of a gene which induces depression and/or is associated with depression are assigned to a gene having an increased expression rate. Reference is made to the exemplary embodiments for further discussion of specific procedures of the method.

It is thus possible to identify further genes in conjunction with depression induced in particular by the use of interferon, or which are related to endogenous depression or cause same.

In particular when the condition is endogenous depression, the method for identifying and/or determining at least one nucleic acid molecule, in particular a gene, which induces depression and/or is associated with depression may comprise the following steps:
(a) Creating a gene expression and/or gene activity profile for a number of test subjects of a test group with endogenous depression;
(b) Analyzing and comparing or balancing the respective gene expression and/or gene activity profiles of (i) test subjects with endogenous depression and (ii) test subjects without endogenous depression; and
(c) Identifying at least one nucleic acid molecule, in particular at least one gene, having increased gene expression and/or gene activity for (i) test subjects with endogenous depression compared to (ii) test subjects without endogenous depression.

The method may include the following step after step (c):
(d) Assigning the nucleic acid molecule, in particular the gene, identified in step (c) as a nucleic acid molecule which induces depression and/or is associated with depression, in particular a gene which induces depression and/or is associated with depression, associated with endogenous depression.

With regard to the method according to the invention corresponding to the present and ninth aspects of the present invention, reference is made to the discussion of the other aspects of the present invention, which correspondingly apply.

As stated above, the identified genes are in particular genes which induce depression and/or are associated with depression, and/or are IFN response genes whose expression is correlated with IFN-induced depression, preferably genes which induce depression and/or are associated with depression. In this regard, within the scope of the present invention genes in particular are used which are selected from the group comprising
DYNLT1, in particular having the transcript ID (locus) NM_{—}006519, in particular according to SEQUENCE LISTING SEQ. ID. NO. 1 and/or Table 1,
MEF2A, in particular having the transcript ID (locus) NM_{—}005587, in particular according to SEQUENCE LISTING SEQ. ID. NO. 2 and/or Table 1,
TOR1B, in particular having the transcript ID (locus) NM_{—}014506, in particular according to SEQUENCE LISTING SEQ. ID. NO. 3 and/or Table 1,
DISC1, in particular having the transcript ID (locus) NM_{—}018662, in particular according to SEQUENCE LISTING SEQ. ID. NO. 4 and/or Table 1,
GCH1, in particular having the transcript ID (locus) NM_{—}000161, in particular according to SEQUENCE LISTING SEQ. ID. NO. 5 and/or Table 1,
ST3GAL5, in particular having the transcript ID (locus) NM_{—}003896, in particular according to SEQUENCE LISTING SEQ. ID. NO. 6 and/or Table 1,
PSMB9, in particular having the transcript ID (locus) NM_{—}002800, in particular according to SEQUENCE LISTING SEQ. ID. NO. 7 and/or Table 1,
GLRX, in particular having the transcript ID (locus) NM_{—}002064, in particular according to SEQUENCE LISTING SEQ. ID. NO. 8 and/or Table 1,
RBCK1, in particular having the transcript ID (locus) NM_{—}006462, in particular according to SEQUENCE LISTING SEQ. ID. NO. 9 and/or Table 1, and
ZNF200, in particular having the transcript ID (locus) NM_{—}003454, in particular according to SEQUENCE LISTING SEQ. ID. NO, 10 and/or Table 1,
STAT1, in particular having the transcript ID (locus) NM_{—}007315, in particular according to SEQUENCE LISTING SEQ. ID. NO. 11 and/or Table 2,
RTP4, in particular having the transcript ID (locus) NM_{—}022147, in particular according to SEQUENCE LISTING SEQ. ID. NO. 12 and/or Table 2,
UBE2L6, in particular having the transcript ID (locus) NM_{—}004223, in particular according to SEQUENCE LISTING SEQ. ID. NO. 13 and/or Table 2,
GBP1, in particular having the transcript ID (locus) NM_{—}002053, in particular according to SEQUENCE LISTING SEQ. ID. NO. 14 and/or Table 2,
CCL8, in particular having the transcript ID (locus) NM_{—}005623, in particular according to SEQUENCE LISTING SEQ. ID. NO. 15 and/or Table 2,
TNFSF10, in particular having the transcript ID (locus) NM_{—}003810, in particular according to SEQUENCE LISTING SEQ. ID. NO. 16 and/or Table 2,
   and combinations thereof.

According to the invention, genes which induce depression and/or are associated with depression are preferably used within the scope of the present invention, whereby the gene or genes are selected from the group comprising
DYNLT1, in particular having the transcript ID (locus) NM_{—}006519, in particular according to SEQUENCE LISTING SEQ. ID. NO. 1 and/or Table 1,
MEF2A, in particular having the transcript ID (locus) NM_{—}005587, in particular according to SEQUENCE LISTING SEQ. ID. NO. 2 and/or Table 1,
TOR1B, in particular having the transcript ID (locus) NM_{—}014506, in particular according to SEQUENCE LISTING SEQ. ID. NO. 3 and/or Table 1,
DISC1, in particular having the transcript ID (locus) NM_{—}018662, in particular according to SEQUENCE LISTING SEQ. ID. NO. 4 and/or Table 1,
GCH1, in particular having the transcript ID (locus) NM_{—}000161, in particular according to SEQUENCE LISTING SEQ. ID. NO. 5 and/or Table 1,
ST3GAL5, in particular having the transcript ID (locus) NM_{—}003896, in particular according to SEQUENCE LISTING SEQ. ID. NO. 6 and/or Table 1,
PSMB9, in particular having the transcript ID (locus) NM_{—}002800, in particular according to SEQUENCE LISTING SEQ. ID. NO. 7 and/or Table I,
GLRX, in particular having the transcript ID (locus) NM_{—}002064, in particular according to SEQUENCE LISTING SEQ. ID. NO. 8 and/or Table 1,
RBCK1, in particular having the transcript ID (locus) NM_{—}006462, in particular according to SEQUENCE LISTING SEQ. ID. NO. 9 and/or Table 1, and
ZNF200, in particular having the transcript ID (locus) NM_{—}003454, in particular according to SEQUENCE LISTING SEQ. ID. NO. 10 and/or Table 1,
   and combinations thereof.

According to the invention, genes which in particular induce depression and/or are associated with depression are particularly preferably used within the scope of the present invention, whereby the gene or genes are selected from the group comprising DYNLT1, MEF2A, TOR1B, DISC1, GCH1, and ST3GAL5, in particular as defined above.

According to a less preferred embodiment according to the invention, used in particular within the scope of the present invention are IFN response genes whose expression is correlated with IFN-induced depression, whereby the gene or genes are selected from the group comprising
STAT1, in particular having the transcript ID (locus) NM_{—}007315, in particular according to SEQUENCE LISTING SEQ. ID. NO. 11 and/or Table 2,
RTP4, in particular having the transcript ID (locus) NM_{—}022147, in particular according to SEQUENCE LISTING SEQ. ID. NO. 12 and/or Table 2,
UBE2L6, in particular having the transcript ID (locus) NM_{—}004223, in particular according to SEQUENCE LISTING SEQ. ID. NO. 13 and/or Table 2,
GBP1, in particular having the transcript ID (locus) NM_{—}002053, in particular according to SEQUENCE LISTING SEQ. ID. NO. 14 and/or Table 2,
CCL8, in particular having the transcript ID (locus) NM_{—}005623, in particular according to SEQUENCE LISTING SEQ. ID. NO. 15 and/or Table 2,
TNFSF10, in particular having the transcript ID (locus) NM_{—}003810, in particular according to SEQUENCE LISTING SEQ. ID. NO. 16 and/or Table 2,
   and combinations thereof.

Furthermore, the depression involves in particular depression related to the treatment and/or therapy of hepatitis, in particular chronic type C hepatitis.

In particular, the depression is a condition that is associated with and/or caused by α-interferon, especially pegylated α-interferon, in particular whereby the depression is associated with and/or caused by the preferably systemic administration of α-interferon, especially pegylated α-interferon, in particular in the treatment and/or therapy of hepatitis, in particular chronic type C hepatitis.

In other words, the depression is a condition that is associated with and/or caused by increased gene activity and/or gene expression of at least one gene as previously defined, in particular whereby the gene activation is associated with and/or caused by preferably systemic administration of α-interferon, especially pegylated α-interferon, in particular in the treatment and/or therapy of hepatitis, in particular chronic type C hepatitis.

As stated above, the depression in question within the scope of the present invention may also be endogenous depression.

Furthermore, the applicant has surprisingly found that for endogenous depression, an increased interferon level or an increased interferon concentration and/or increased interferon production may be present in the affected patients. In this regard the applicant has been able to demonstrate in studies that for patients with endogenous depression, an increased level or an increased interferon concentration and/or increased interferon production, i.e., (bio-)synthesis of endogenous interferon, in particular α-interferon (IFN-α), such as α-1-interferon (IFN-α-1) and/or α-2-interferon (IFN-α-2), β-interferon (IFN-β), and/or γ-interferon (IFN-γ), is present, as illustrated in the exemplary embodiments. The applicant has surprisingly found that the occurrence of endogenous depression, in particular accompanied by a severe depressive episode (SDE), may also be caused by an increased level or concentration and/or increased production of endogenous interferon, in particular of the aforementioned type. In this regard—with no intent of identifying a theoretical basis—the occurrence of endogenous depression which is correlated with or accompanied by the presence of an increased level or an increased concentration and/or production of endogenous interferon, in particular of the aforementioned type, may be explained by the fact that the increased interferon level or the increased concentration and/or production of endogenous interferon sometimes causes increased gene activity, in particular in genes which induce depression and/or are associated with depression, for example as described above.

Accordingly, the present invention further relates to finding and/or providing substances which modulate, in particular prevent or at least reduce, the physiological activity of endogenous interferon, in particular α-interferon, such as α-1-interferon and/or α-2-interferon, β-interferon, and/or γ-interferon. Such substances may be used as therapeutic agents in preventative and/or curative treatment of endogenous depression. This may involve, for example, a substance which reduces or suppresses the production or (bio)synthesis of interferon, in particular of the aforementioned type. The substance which reduces/or suppresses the production or (bio)synthesis of interferon may be such that the substance intervenes in the interferon synthesis pathway or regulates same or interacts with components of the interferon synthesis pathway, thereby preventing or at least reducing the endogenous formation of interferon, in particular of the aforementioned type. In this regard this may also involve, for example, a gene-regulating substance which in particular modulates, in particular reduces, the gene activity of genes involved in the (bio)synthesis of interferon. Similarly, this subject matter of the present invention also concerns finding and/or providing substances which modulate, in particular prevent, the interferon signal pathway. In this regard, the substance itself may interact, for example, with the endogenous interferon, in particular α-interferon, such as IFN-α-1 and/or IFN-α-2, and/or IFN-γ, in such a way that the activity of the interferon is reduced or inhibited. Thus, the substances may be interferon blockers, for example. For example, the substances may also be compounds which, for example, react with interferon receptors, for example in the manner of an interferon receptor blocker.

The present invention similarly relates to the use of the substances described above which modulate, in particular inhibit, the activity of endogenous interferon, in particular α-interferon, such as IFN-α-1 and/or IFN-α-2, IFN-β, and/or IFN-γ, for producing a medicament for the preventative and/or curative treatment of endogenous depression.

Likewise, the present invention further relates to a method for determining the risk of a test subject of developing endogenous depression, whereby an increased risk of developing endogenous depression is assigned to the test subject for the case that the test subject has an increased endogenous interferon level or an increased concentration of endogenous interferon and/or increased production of interferon, in particular α-interferon, such as IFN-α-1 and/or IFN-α-2, IFN-β, and/or IFN-γ.

Within the scope of the present invention, as a whole it has been possible to provide a novel approach to the treatment of depression, in particular of the type defined above, which in particular has a genetic basis. Within the scope of the present invention, it has surprisingly been possible to identify genes which cause depression, in particular in patients treated with α-interferon and which thus represent an ideal target for the treatment or avoidance of depression.

On this basis, within the scope of the present invention possibilities are provided for early detection of depression as well as for treatment of depression, whereby the depression in particular may be interferon-induced depression as well as endogenous depression. As stated above, by use of the method according to the invention other genes which induce depression and/or are associated with depression may be identified. The genes identified in this manner assist in predicting depression at the beginning of treatment or avoiding the development of depression, in particular for patients treated with interferon. This is of great significance, since approximately 30% of patients treated with interferon develop depression. In addition, the identified genes which induce depression and/or are associated with depression may play a role in the pathogenesis of endogenous depression, so that according to the invention targeted pharmacological suppression of its activity may be achieved.

Further embodiments, modifications, variations, and advantages of the present invention may be readily recognized and implemented by one skilled in the art in reading the description section without departing from the scope of the present invention.

The present invention is illustrated with reference to the following exemplary embodiments which, however, in no way limit the present invention.

### Exemplary Embodiments

### I. Studies on Patients with Hepatitis C

On the basis of the studies described below it is possible to identify relevant risk factors for IFN-induced depression in order to develop preventative treatment strategies. For this purpose, the primary transcriptional response to IFNα-2a plus ribavirin was investigated in patients with hepatitis C. In this manner, candidate genes may be identified whose expression is associated with IFN-induced severe depression.

### Patients and Methods:

A total of 50 patients of Caucasian origin with histologically verified chronic hepatitis C were treated with a standard combination therapy composed of pegylated α-interferon IFNα-2a (Pegasys, 180 μg once per week) over 12 months (HCV genotype 1, n=40) or 6 months (HCV genotype 2/3, n=3/7) in combination with ribavirin (800-1200 mg daily). RNA was isolated from peripheral blood (PAXgen, PreAnalytiX) which had been withdrawn 12 hours before and 12 hours after the first injection. The gene expression profiles of approximately 22,000 human genes were determined using DNA chips (HG-U133A 2.0, Affymetrix). After normalizing the chip raw data, by means of significance and class prediction analyses genes were identified which are differentially regulated in patients with and without IFN-/IFNα-induced depression. The expression of these genes was validated by quantitative real-time RT-PCR. The patients were psychologically evaluated in cooperation with the Essen Rhine Clinics, Clinic for Psychosomatic Medicine and Psychotherapy. Using Mini-DIPS (Diagnostic Interview for Psychological Disorders) and psychometric instruments such as the Beck Depression Inventory, the development of IFN-induced depression in conjunction with treatment was qualitatively and quantitatively analyzed on a quarterly basis.

### Results:

11 of 50 patients (22%) suffered from IFN-induced depression. 11 randomly selected non-depressed patients were included in a comparative analysis. As a result of the class prediction analysis it was possible to predict IFN-induced depression with an accuracy of 91%, using 16 genes. Of these genes, 6 represent typical IFN-stimulated genes (ISG) or IFN response genes. Of these 16 genes, 6 were also genes for which a correlation with recurring severe depression or neuronal development processes in the brain had been published (“depression gene”). For all 16 genes, the transcriptional response to IFN in patients with IFN-induced depression was more pronounced than in the comparative group without clinical depression. Thus, the gene response in patients with IFN-induced depression was consistently more intense than in the comparative group. The basal expression of the IFN response genes (before IFN injection) was significantly decreased in the majority of patients with IFN-induced depression. The basal expression of the 6 “depression genes” showed no significant differences in either group. These findings were verified by quantitative real-time polymerase chain reaction (RT-PCR).

### Conclusions:

The data indicate original involvement of genes of the interferon response in the development of severe depression which frequently occurs as a side effect in antiviral treatment with IFN-α. Differences in the response behavior of these genes allow predictions concerning the manifestation of severe depression already present in the initial phase of treatment. The functional analysis of the identified genes could facilitate the development of new medications for improved compatibility and thus improved efficiency of the HCV treatment. In addition, the genes identified by the present inventors may also be important for the pathogenesis, and therefore the treatment, of endogenous depression not primarily associated with IFN.

### Detailed Description of the Methods:

### 1. Sampling and Isolation of Total RNA:

After obtaining written patient consent for routine examinations, approximately 10 mL of blood was withdrawn from the arm veins of each patient 12 hours before and 12 hours after the first IFNα injection. PAXgene Blood RNA tubes (PreAnalytiX, Becton Dickinson, Heidelberg) were used to stabilize the RNA in the peripheral blood cells directly upon blood withdrawal. The RNA was then prepared using the PAXgene Blood RNA Kit (PreAnalytiX, Qiagen, Hilden) according to the manufacturer's instructions. In brief: The PAXgene vials were centrifuged (10 min, 4000 rpm, 20° C.), the supernatant was suctioned off, and the pellet was resuspended in 5 mL RNase-free water (kit). After recentrifugation (10 min, 4000 rpm, 20° C.) the supernatant was suctioned off and discarded. The pellet was resuspended in 360 μL BR1 buffer (kit) and transferred to an RNase-free 1.5-mL vial (kit). After adding 300 μL, BR2 buffer (kit) and 40 μL proteinase K (kit), the sample was mixed and incubated for 15 min at 55° C. (heating block). The lysate (approximately 700 μL) was then loaded on the “shredder” column (kit) and centrifuged (3 min, 13,000 rpm, 20° C.). The supernatant was then transferred to a new RNase-free 1.5-mL vial (kit), 360 μL ethanol (absolute) was pipetted in, and the sample was thoroughly mixed. One PAXgen column per sample was loaded with 700 μL of the lysate and centrifuged (1 min, 10,000 rpm, 20° C.). The flow-through and the collection tubes were discarded, the column was placed in a new collection tube (kit), loaded with the remainder of the sample, and recentrifuged (1 min, 10,000 rpm, 20° C.). The flow-through and the collection tubes were discarded, and the column was placed in a new collection tube (kit) and successively washed, once with BR3 buffer (kit) and twice with BR4 buffer (kit). The flow-through and the collection tubes were discarded, and the column was placed in an elution tube (kit) and washed with 45 μl BR5 buffer (kit). The eluate was reloaded on the column and centrifuged (1 min, 10,000 rpm, 20° C.). Four samples per withdrawal period for each patient (approximately 180 μL) were purified.

The RNA was further concentrated for the subsequent chip analyses, using the RNeasy MinElute Cleanup Kit (Qiagen, Hilden). In brief: the samples were adjusted to 200 μL using RNase-free water. 700 μL RLT buffer (kit) and 500 μL ethanol (absolute) were pipetted in. One column per sample was loaded with 700 μL sample and centrifuged (15 s, 10,000 rpm, 20° C.). Each column was then washed with 500 μL RPE buffer (kit). 500 μL ethanol (80%) was pipetted in and centrifuged (2 min, 10,000 rpm, 20° C.). The flow-through and the collection tubes were discarded, the column was placed in a new collection tube (kit), and the column was centrifuged and dried with the cover open (5 min, 13,000 rpm, 20° C.). The flow-through and the collection tubes were discarded, and the column was placed in an elution tube (kit) and washed with 18 μL BR5 buffer (kit) (1 min, 13,000 rpm, 20° C.). The sample was incubated for 5 min at 65° C. (heating block), then cooled on ice and stored at −80° C.

### 2. Analysis of Gene Expression Using Affymetrix Genechips (Oligonucleotide Microarray):

The expression analyses on Affymetrix GeneChips were performed in cooperation with the local BioChip laboratory (Institute for Cell Biology, Essen Uniclinic). First, the total RNA was transformed into double-stranded cDNA. For the synthesis of the first cDNA strand, for each sample 8 μL (10 μg) total RNA together with 1 μL of a mixture of 3 poly-A control RNA and 1 μL (100 μM) T7-oligo-d(T)24 primer (MWG Biotech, Munich) were incubated for 10 min at 70° C. (heating block) and then transferred to ice. For this purpose, 4 μL “first strand” buffer (5×), 2 μL (0.1 M) DTT, and 1 μL (10 mM) dNTP mix were pipetted in and incubated for 2 min at 42° C. (heating block). 2 μL (200 units) Superscript II (Life Technologies, Karlsruhe) was then added, and the batch was incubated for 1 hour at 42° C.

For the synthesis of the second cDNA strand the following mixture was pipetted in: 30 μL “second strand” buffer (5×), 91 μL RNase-free water, 3 μL (10 mM) dNTP mix, 4 μL (40 units) Escherichia coli DNA polymerase I (Life Technologies), 1 μL (12 units) E. coli DNA ligase (TaKaRa, Gennevilliers, France), and 1 μL (2 units) RNase H (TaKaRa). This reaction mixture was incubated for 2 hours at 16° C. (coolable thermoblock). 2.5 μL (10 units) T4 DNA polymerase I (TaKaRa) was then added, followed by incubation for 5 min at 16° C.

The reaction was then terminated by adding 10 μL (0.5 M) EDTA, and the double-stranded cDNA was extracted with phenol/chloroform. The aqueous phase was separated (Phase Lock Gel separation, Eppendorf, Hamburg). After precipitation the cDNA was dissolved in 12 μL RNase-free water. The biotinylated cRNA was synthesized using the Bio-Array High Yield RNA Transcript Labeling Kit (Enzo Diagnostics, NY, USA) according to the manufacturer's instructions. The labeled cRNA was purified using the RNeasy Mini Kit (Qiagen). Fragmentation of the cRNA, hybridization, and washing, staining, and scanning of the GeneChips (HG-U133A 2.0, Affymetrix, Santa Clara, USA) were performed in the GeneArray Scanner 2500 (Agilent, Palo Alto, USA) according to the manufacturer's instructions (Technical Manual, Affymetrix).

### 3. Analysis of the GeneChip Data:

The processing of the scanner images, calculation of the signals, and comparative analyses of the sample pairs (before and after IFN injection) were performed with GeneChip Operating Software (GCOS v1.2, Affymetrix), using the MASS algorithm. Further analyses and data filtering were carried out using Data Mining Tools v3.1 (Affymetrix). Evaluation of raw data using GCOS in the individual chip analysis for each gene resulted in a so-called “detection call,” which is an evaluation of the sample concerning whether the particular gene is active (P=present) or inactive (A=absent). In the comparative chip analysis, data for the sample after the IFN injection were compared to the associated data for the same patient before the IFN injection (baseline). After these comparative analyses GCOS calculated the so-called “change call,” which is an evaluation of the sample concerning whether the particular gene is increased (I), decreased (D), or not changed (NC) after IFN injection. To reduce the quantity of data to a manageable level, to begin with only genes were filtered out which were increased (“I call”) or decreased (“D call”) in at least one patient. Of 22,216 genes, 8,093 genes then remained. The following were used as additional gene groups in further statistical analyses: a) I or D call in more than one patient (4,236 genes), b) I or D call in more than 25% of the patients (1,342 genes), and c) I or D call in more than 50% of the patients (586 genes).

To minimize individual experimental deviations, the data for all GeneChips were normalized using the robust multiarray average (RMA) method. For this purpose, background adjustment and quantile normalization of the chip raw data were performed using RMAExpress v0.4.1 (http://rmaexpress.bmbolstad.com). Further analyses were [carried out] using a spreadsheet (Excel 2003, Microsoft). Two-class analyses were performed to identify candidate genes which are differentially regulated in the two clinical groups of patients with and without IFN-induced depression. On the one hand, there was a significant difference in the genes that were identified, using Excel Add-In SAM v3.0 (SAM=Significance Analysis of Microarrays, http://www.stat.stanford.edu/˜tibs/SAM). On the other hand, Excel Add-In PAM v2.1 (PAM=Prediction Analysis of Microarrays, http://www.stat.stanford.edu/˜tibs/PAM) was used to investigate whether it was possible to predict membership in one of the two clinical groups, and the number of genes necessary for this purpose. GraphPad Prism v4.03 (GraphPad Software, San Diego, USA) was used for additional statistical analyses (correlation, Wilcoxon test, ANOVA) and for creating graphics.

### 4. Validation of Expression Data by Quantitative RT-PCR:

To check the gene expression data from the GeneChip experiments, the mRNA of individual candidate genes was determined before and after the IFN injection, using quantitative real-time polymerase chain reaction (PCR). Up to 36 reaction vials (0.2 μL, NerbePlus, Winsen/Luhe) were measured in the Rotor-Gene 2000 real-time amplification system (Corbett Research, Mortlake, Australia). Transcription of the RNA into cDNA and subsequent amplification (including real-time detection) were performed as quantitative one-step reverse transcriptase PCR (qRT-PCR), using the QuantiTect SYBR Green RT-PCR Kit (Qiagen) according to the manufacturer's instructions. In brief: 2 μL (20-200 ng) of each RNA sample was pipetted into 23 μL RT-PCR mix composed of 12.5 μL RT Master Mix, 0.3 μL RT mix, 7.7 μL RNase-free water, and 2.5 μL (0.5 μM) primer (QuantiTect Primer Assay, Qiagen). The samples were first incubated in the Rotor-Gene 2000 for 30 min at 50° C. (cDNA reaction), followed by 15 min at 95° C. (inactivation of reverse transcriptase, activation of Hot Start Taq Polymerase). The samples were amplified over 35 to 40 cycles, in each case for 20 sec at 95° C., 20 sec at 55° C., and 40 sec at 72° C. A melting curve was plotted after the last cycle for evaluation of the specificity of the reaction. The temperature was increased in 0.5° C. increments from 72 to 94° C., with a fluorescence measurement (510 nm wavelength) after each temperature increment. During the amplification the fluorescence measurement was performed after each cycle, at the end of the 72° C. phase. For each primer pair used, in addition to the patient samples negative controls (RNase-free water instead of RNA) were included in each PCR run.

For each PCR assay the number of mRNA copies was calculated using standard curves. To take differences in the initial RNA concentration into account, for each RNA sample the mRNA concentration was determined for β-actin, a housekeeping gene which is continuously expressed under IFNα stimulation.

TABLE 1
Genes which induce depression and/or are associated with depression:

| Gene | | Transcript | | Entrez | |
|---|---|---|---|---|---|
| symbol | Gene name | ID | Localization | Gene | MIM |
| DYNLT1 | Dynein, light chain, Tctex- | NM_006519 | 6q25.2-q25.3 | 6993 | 601554 |
| | type 1 | | | | |
| MEF2A | MADS box transcription | NM_005587 | 15q26 | 4205 | 600660 |
| | enhancer factor 2, | | | | |
| | polypeptide A | | | | |
| | (myocyte enhancer factor | | | | |
| | 2A) | | | | |
| TOR1B | Torsin family 1, member B | NM_014506 | 9q34 | 27348 | 608050 |
| | (torsin B) | | | | |
| DISC1 | Disrupted in schizophrenia 1 | NM_018662 | 1q42.1 | 27185 | 605210 |
| GCH1 | GTP cyclohydrolase 1 | NM_000161 | 14q22.1-q22.2 | 2643 | |
| | (dopa- | | | | |
| | responsive dystonia) | | | | |
| ST3GAL5 | ST3 beta-galactoside | NM_003896 | 2p11.2 | 8869 | |
| | alpha-2,3- | | | | |
| | sialyltransferase 5 | | | | |
| PSMB9 | Proteasome (prosome, | NM_002800 | 6p21.3 | 5698 | |
| | macropain) subunit, beta | | | | |
| | type, 9 (large | | | | |
| | multifunctional peptidase | | | | |
| | 2) | | | | |
| GLRX | Glutaredoxin | NM_002064 | 5q14 | 2745 | |
| | (thioltransferase) | | | | |
| RBCK1 | RanBP-type and C3HC4- | NM_006462 | 20p13 | 10616 | |
| | type | | | | |
| | zinc finger containing 1 | | | | |
| ZNF200 | Zinc finger protein 200 | NM_003454 | 16p13.3 | 7752 | |

TABLE 2
IFN response genes or IFN-stimulated genes (ISG) whose
expression is correlated with IFN-induced depression:

| Gene | | Transcript | |
|---|---|---|---|
| symbol | Gene name | ID | Localization |
| STAT1 | Signal transducer and activator of transcription, | NM_007315 | 2q32.2 |
| | 1.91 kDa | | |
| RTP4 | Receptor transporter protein 4 | NM_022147 | 3q27.3 |
| UBE2L6 | Ubiquitin-conjugating enzyme E2L 6 | NM_004223 | 11q12 |
| GBP1 | Guanylate binding protein 1, interferon- | NM_002053 | 1p22.2 |
| | inducible, 67 kDa | | |
| CCL8 | Chemokine (C-C motif) ligand 8 | NM_005623 | 17q11.2 |
| TNFSF10 | Tumor necrosis factor (ligand) superfamily, | NM_003810 | 3q26 |
| | member 10 | | |

### II. Studies on Psychiatric Patients and Comparison to Patients with Hepatitis C

For validation of the genes associated with depression, 23 psychiatric patients in in-patient treatment due to a severe depressive episode (SDE) were prospectively examined (Table 3). All of the patients were receiving antidepressant medication at the time that the molecular-biological tests were performed. Diagnosis of an SDE was based on a psychiatric evaluation by a psychiatrist. The severity of the depression was determined using the Hamilton Depression Rating Scale (HAMD-17). Recurrent depressive disorder (ICD-10: F33.2) was diagnosed in 18 patients, one of whom also exhibited psychotic symptoms (ICD-10: F33.3). Three patients had bipolar affective disorder with a severe depressive episode (ICD-10: F31.4), and two patients exhibited severe depressive episode (ICD-10: F32.2). Eleven healthy test subjects with no clinical symptoms of depressive disorder were used as controls.

Peripheral blood (PBMC) samples were withdrawn from healthy test subjects and psychiatric patients and stimulated in vitro with 100 U/mL pegylated IFN-α-2a for 16 hours. The expression of ISGs as well as selected genes was determined before or after stimulation.

The following additional methods were used:

1. RNA Isolation from Cultured PBMC:

Total RNA was isolated from cells, using TRIzol (Invitrogen, Karlsruhe, Germany), and was purified by DNase digestion of residual genomic DNA using the RNeasy Mini Kit and the RNase-Free DNase Set (both from Qiagen).

### 2. Real-Time Detection of Ifn Gene Transcripts Using One-Step RT-PCR:

One-step RT-PCR using real-time detection was performed using the Rotor-Gene 2000 real-time amplification system (Corbett Research, Mortlake, Australia) and the QuantiTect SYBR Green RT-PCR Kit (Qiagen, Hilden, Germany). For each mRNA the number of copies was compared to the number of β-actin transcripts.

### 3. Isolation and In Vitro Stimulation of PBMC:

PBMC was isolated from heparinized blood by centrifugation over a Ficoll density gradient (Lymphocyte Separation Medium 1077, PAA, Coelbe) at 350 g for 20 min. The cells were transferred into 6-well plates (Greiner, Niirtingen) in a concentration of 2×10⁶ cells/well and cultured in medium (RPMI 1640, PAA) containing 2% pooled human AB serum, 2 mM L-glutamine, and 50 μg/mL gentamicin at 37° C. and 5% CO₂. A medium control without stimulus was included in all experiments.

### Results:

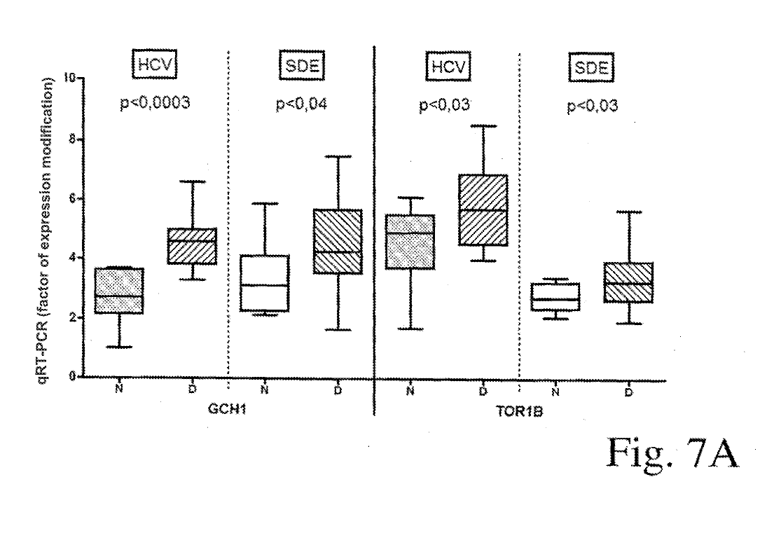

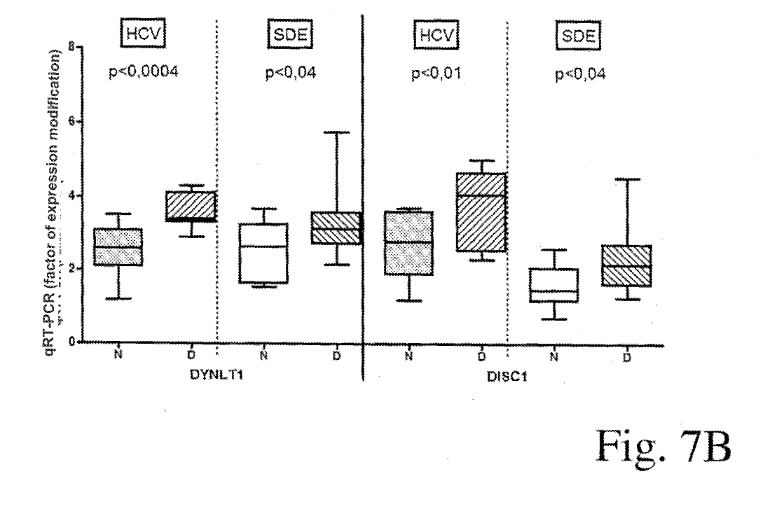

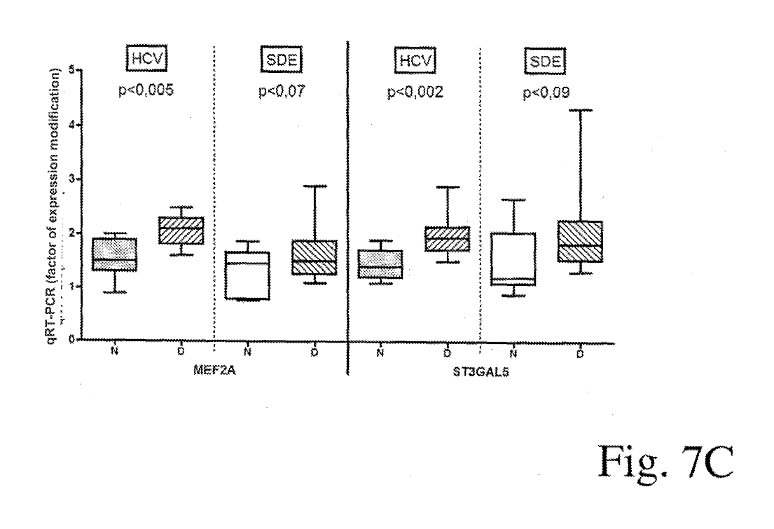

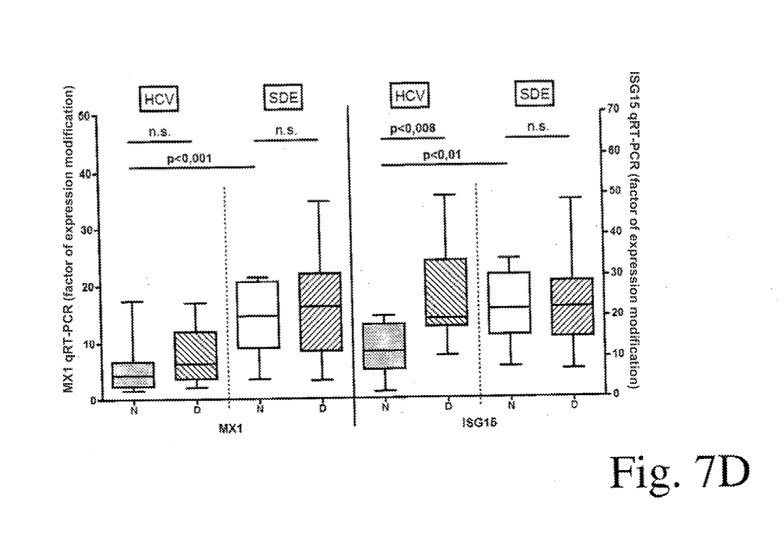

In patients with an SDE, pegylated IFN-α-2a resulted in significantly higher induction of the target genes GCH1, TOR1B, DYNLT1, and DISC1, and a tendency toward higher induction of MEF2A and ST3GAL5. No differences were found for conventional ISGs such as MX1 or ISG15 (FIG. 7D), as well as IFIT1 and IF116 (not shown). It was thus concluded that for patients with an SDE, selective, not general, enhanced stimulation by type I interferons was present.

In this regard, FIGS. 7A through 7D show the IFN-stimulated expression of genes in patients with HCV on the one hand, and psychiatric patients with an SDE on the other hand. Total RNA of HCV patients with (n=11) or without (n=11) consecutive IFN-induced depression was isolated 12 hours before and 12 hours after the first injection of pegylated IFN-α-2a. For validation of these data in an independent cohort, PBMC from 11 healthy controls and 22 patients with SDE was isolated and stimulated in vitro with 100 U/mL pegylated IFN-α-2a for 16 hours. The gene expression was analyzed by quantitative RT-PCR. The data are shown as box plots (range, 25% and 75% percentile, mean).

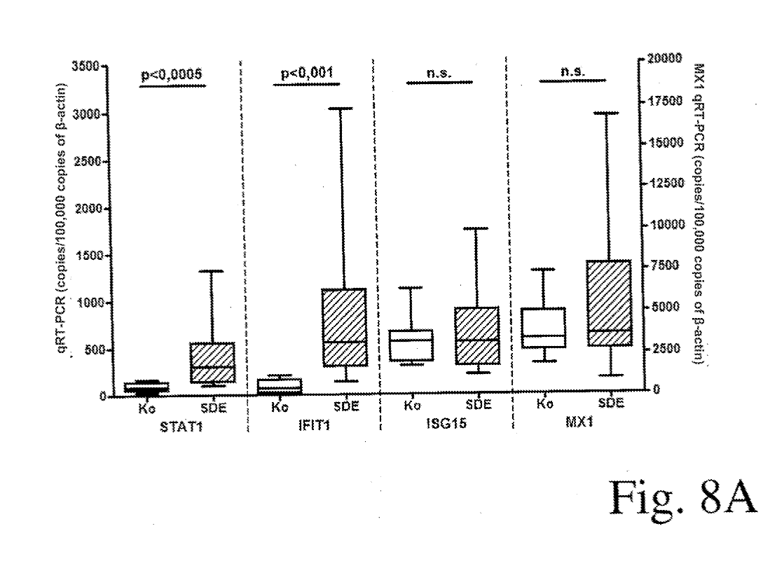

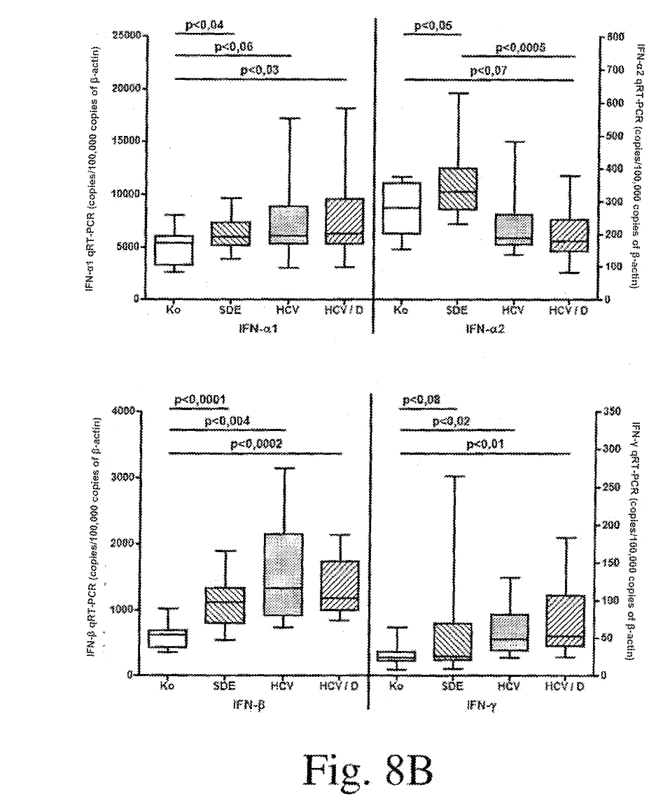

Analysis of the gene expression without prior IFN stimulation in patients with SDE showed significant upregulation of conventional ISGs such as STAT1 and IFIT1 in comparison to healthy controls (FIG. 8A), which was attributable to increased production of endogenous IFNα in these patients. To test this hypothesis, basal values of the most common IFN-α subtypes (IFN-α-1 and IFN-α-2), IFN-β, and IFN-α were determined by quantitative RT-PCR (FIG. 8B). Distinct upregulation of IFN-β production was seen in patients with SDE in comparison to healthy controls. Increased production of IFN-α-1 and IFN-α-2 was also found, as well as a trend toward greater production of IFN-γ.

In this regard, FIGS. 8A and 8B show increased gene expression and IFN production in psychiatric patients with an SDE. Total RNA was isolated from HCV patients with (n=11) or without (n=11) consecutive IFN-induced depression, 11 healthy controls, and 22 patients with an SDE. The basal gene expression (FIG. 8A), and the expression of IFN-α-1 and IFN-α-2 (upper two diagrams in FIG. 8B) and IFN-β and IFN-γ (lower two diagrams in FIG. 8B) were analyzed by quantitative RT-PCR. The data are shown as box plots (range, 25% and 75% percentile, mean).

TABLE 3
Patient data

| | HCV | HCV | Depressive |
|---|---|---|---|
| | No depression | Depression | episode |
| Sex [F/M] | 15/124 | 4/7 | 14/8 |
| Age [years, mean ± S.D.] | 44.9 ± 10.9 | 38.5 + 6.1 | 51.2 ± 13.6 |
| Weight [kg, mean ± S.D.] | 76.4 ± 14.3 | 76.1 ± 12.8 | 73.9 + 13.4 |
| BMI [mean ± S.D.] | 25.6 ± 4.3 | 25.3 ± 3.7 | 25.4 ± 4.3 |
| GPT [U/mL mean ± S.D.] | 106.9 ± 89.3 | 68.6 ± 32.5 | 25.5 ± 13.9 |
| Percent cirrhosis | 33% | 25% | n.d. |
| HCV genotypes [1/2/3/4] | 32/1/5/1 | 8/0/3/0 | n.d. |
| HCV RNA basal value | 1,254,687 ± 226,217 | 1,001,580 ± 349,957 | n.d. |
| [copies/mL, mean ± SEM] | | | |
| ETR [%] | 48.7 | 45.5 | n.d. |
| SVR [%] | 30.8 | 36.4 | n.d. |

1-31. (canceled)

32. A method for determining the risk of a test subject of developing depression and for predicting individual effects and side-effects of medicaments, namely depression as a result of interferon therapy, wherein an elevated risk of developing depression and altered drug effects and side-effects are assigned to the test subject for the case that the test subject has increased gene activity or increased gene expression of at least one gene which induces depression or is associated with depression.

33. The method according to claim 32, wherein the medicament is alpha-interferon administered within the scope of treatment and therapy of hepatitis.

34. The method according to claim 32, wherein the gene is selected from the group comprising:
DYNLT1 having the transcript ID (locus) NM_{—}006519 according to SEQUENCE LISTING SEQ. ID. NO. 1 and Table 1,
MEF2A having the transcript ID (locus) NM_{—}005587 according to SEQUENCE LISTING SEQ. ID. NO. 2 and Table 1,
TOR1B having the transcript ID (locus) NM_{—}014506 according to SEQUENCE LISTING SEQ. ID. NO. 3 and Table 1,
DISC1 having the transcript ID (locus) NM_{—}018662 according to SEQUENCE LISTING SEQ. ID. NO. 4 and Table 1,
GCH1 having the transcript ID (locus) NM_{—}000161 according to SEQUENCE LISTING SEQ. ID. NO. 5 and Table 1,
ST3GAL5 having the transcript ID (locus) NM_{—}003896 according to SEQUENCE LISTING SEQ. ID. NO. 6 and Table 1,
PSMB9 having the transcript ID (locus) NM_{—}002800 according to SEQUENCE LISTING SEQ. ID. NO. 7 and Table 1,
GLRX having the transcript ID (locus) NM_{—}002064 according to SEQUENCE LISTING SEQ. ID. NO. 8 and Table 1,
RBCK1 having the transcript ID (locus) NM_{—}006462 according to SEQUENCE LISTING SEQ. ID. NO. 9 and Table 1,
ZNF200 having the transcript ID (locus) NM_{—}003454 according to SEQUENCE LISTING SEQ. ID. NO, 10 and Table 1,
STAT1 having the transcript ID (locus) NM_{—}007315 according to SEQUENCE LISTING SEQ. ID. NO. 11 and Table 2,
RTP4 having the transcript ID (locus) NM_{—}022147 according to SEQUENCE LISTING SEQ. ID. NO. 12 and Table 2,
UBE2L6 having the transcript ID (locus) NM_{—}004223 according to SEQUENCE LISTING SEQ. ID. NO. 13 and Table 2,
GBP1 having the transcript ID (locus) NM_{—}002053 according to SEQUENCE LISTING SEQ. ID. NO. 14 and Table 2,
CCL8 having the transcript ID (locus) NM_{—}005623 according to SEQUENCE LISTING SEQ. ID. NO. 15 and Table 2,
TNFSF10 having the transcript ID (locus) NM_{—}003810 according to SEQUENCE LISTING SEQ. ID. NO. 16 and Table 2,
and combinations thereof.

35. The method according to claim 32, wherein the gene is selected from the group comprising:
DYNLT1 having the transcript ID (locus) NM_{—}006519 according to SEQUENCE LISTING SEQ. ID. NO. 1 and Table 1,
MEF2A having the transcript ID (locus) NM_{—}005587 according to SEQUENCE LISTING SEQ. ID. NO. 2 and Table 1,
TOR1B having the transcript ID (locus) NM_{—}014506 according to SEQUENCE LISTING SEQ. ID. NO. 3 and Table 1,
DISC1 having the transcript ID (locus) NM_{—}018662 according to SEQUENCE LISTING SEQ. ID. NO. 4 and Table 1,
GCH1 having the transcript ID (locus) NM_{—}000161 according to SEQUENCE LISTING SEQ. ID. NO. 5 and Table 1,
ST3GAL5 having the transcript ID (locus) NM_{—}003896 according to SEQUENCE LISTING SEQ. ID. NO. 6 and Table 1,
PSMB9 having the transcript ID (locus) NM_{—}002800 according to SEQUENCE LISTING SEQ. ID. NO. 7 and Table I,
GLRX having the transcript ID (locus) NM_{—}002064 according to SEQUENCE LISTING SEQ. ID. NO. 8 and Table 1,
RBCK1 having the transcript ID (locus) NM_{—}006462 according to SEQUENCE LISTING SEQ. ID. NO. 9 and Table 1, and
ZNF200 having the transcript ID (locus) NM_{—}003454 according to SEQUENCE LISTING SEQ. ID. NO. 10 and Table 1,
and combinations thereof.

36. The method according to claim 32, wherein the gene is selected from the group comprising:
STAT1 having the transcript ID (locus) NM_{—}007315 according to SEQUENCE LISTING SEQ. ID. NO. 11 and Table 2,
RTP4 having the transcript ID (locus) NM_{—}022147 according to SEQUENCE LISTING SEQ. ID. NO. 12 and Table 2,
UBE2L6 having the transcript ID (locus) NM_{—}004223 according to SEQUENCE LISTING SEQ. ID. NO. 13 and Table 2,
GBP1 having the transcript ID (locus) NM_{—}002053 according to SEQUENCE LISTING SEQ. ID. NO. 14 and Table 2,
CCL8 having the transcript ID (locus) NM_{—}005623 according to SEQUENCE LISTING SEQ. ID. NO. 15 and Table 2,
TNFSF10 having the transcript ID (locus) NM_{—}003810 according to SEQUENCE LISTING SEQ. ID. NO. 16 and Table 2,
and combinations thereof.

37. A method for identifying and determining at least one nucleic acid molecule which induces depression or is associated with depression, namely a gene which induces depression or is associated with depression in conjunction with the administration of interferon or a gene which induces depression or is associated with depression in conjunction with endogenous depression, wherein the method comprises the following steps:
(a) Creation of a gene expression profile or a gene activity profile for a number of test subjects of a test group treated with interferon;
(b) Analysis and comparison or balancing of the respective gene expression or gene activity profiles of (i) test subjects who develop depression as the result of interferon therapy and (ii) test subjects who do not develop depression as the result of interferon therapy; or in the case of endogenous depression, analysis and comparison or balancing of the respective gene expression or gene activity profiles of (i) test subjects with endogenous depression and (ii) test subjects without endogenous depression; and
(c) Identification of at least one nucleic acid molecule, namely at least one gene, having increased gene expression or increased gene activity in (i) test subjects with depression compared to (ii) test subjects without depression.

38. The method according to claim 37, wherein the method includes the following step after step (c):
(d) Assignment of the nucleic acid molecule, namely the gene, identified in step (c) as a nucleic acid molecule which induces depression or is associated with depression, said gene selected from a gene which induces depression or is associated with depression in conjunction with the administration of interferon; and in the case of endogenous depression.

39. The method according to claim 37, wherein interferon is administered in conjunction with the treatment of hepatitis.

40. The method according to claim 37, wherein the gene is selected from the group comprising:
DYNLT1 having the transcript ID (locus) NM_{—}006519 according to SEQUENCE LISTING SEQ. ID. NO. 1 and Table 1,
MEF2A having the transcript ID (locus) NM_{—}005587 according to SEQUENCE LISTING SEQ. ID. NO. 2 and Table 1,
TOR1B having the transcript ID (locus) NM_{—}014506 according to SEQUENCE LISTING SEQ. ID. NO. 3 and Table 1,
DISC1 having the transcript ID (locus) NM_{—}018662 according to SEQUENCE LISTING SEQ. ID. NO. 4 and Table 1,
GCH1 having the transcript ID (locus) NM_{—}000161 according to SEQUENCE LISTING SEQ. ID. NO. 5 and Table 1,
ST3GAL5 having the transcript ID (locus) NM_{—}003896 according to SEQUENCE LISTING SEQ. ID. NO. 6 and Table 1,
PSMB9 having the transcript ID (locus) NM_{—}002800 according to SEQUENCE LISTING SEQ. ID. NO. 7 and Table 1,
GLRX having the transcript ID (locus) NM_{—}002064 according to SEQUENCE LISTING SEQ. ID. NO. 8 and Table 1,
RBCK1 having the transcript ID (locus) NM_{—}006462 according to SEQUENCE LISTING SEQ. ID. NO. 9 and Table 1,
ZNF200 having the transcript ID (locus) NM_{—}003454 according to SEQUENCE LISTING SEQ. ID. NO, 10 and Table 1,
STAT1 having the transcript ID (locus) NM_{—}007315 according to SEQUENCE LISTING SEQ. ID. NO. 11 and Table 2,
RTP4 having the transcript ID (locus) NM_{—}022147 according to SEQUENCE LISTING SEQ. ID. NO. 12 and Table 2,
UBE2L6 having the transcript ID (locus) NM_{—}004223 according to SEQUENCE LISTING SEQ. ID. NO. 13 and Table 2,
GBP1 having the transcript ID (locus) NM_{—}002053 according to SEQUENCE LISTING SEQ. ID. NO. 14 and Table 2,
CCL8 having the transcript ID (locus) NM_{—}005623 according to SEQUENCE LISTING SEQ. ID. NO. 15 and Table 2,
TNFSF10 having the transcript ID (locus) NM_{—}003810 according to SEQUENCE LISTING SEQ. ID. NO. 16 and Table 2,
and combinations thereof.

41. The method according to claim 37, wherein the gene is selected from the group comprising:
DYNLT1 having the transcript ID (locus) NM_{—}006519 according to SEQUENCE LISTING SEQ. ID. NO. 1 and Table 1,
MEF2A having the transcript ID (locus) NM_{—}005587 according to SEQUENCE LISTING SEQ. ID. NO. 2 and Table 1,
TOR1B having the transcript ID (locus) NM_{—}014506 according to SEQUENCE LISTING SEQ. ID. NO. 3 and Table 1,
DISC1 having the transcript ID (locus) NM_{—}018662 according to SEQUENCE LISTING SEQ. ID. NO. 4 and Table 1,
GCH1 having the transcript ID (locus) NM_{—}000161 according to SEQUENCE LISTING SEQ. ID. NO. 5 and Table 1,
ST3GAL5 having the transcript ID (locus) NM_{—}003896 according to SEQUENCE LISTING SEQ. ID. NO. 6 and Table 1,
PSMB9 having the transcript ID (locus) NM_{—}002800 according to SEQUENCE LISTING SEQ. ID. NO. 7 and Table I,
GLRX having the transcript ID (locus) NM_{—}002064 according to SEQUENCE LISTING SEQ. ID. NO. 8 and Table 1,
RBCK1 having the transcript ID (locus) NM_{—}006462 according to SEQUENCE LISTING SEQ. ID. NO. 9 and Table 1, and
ZNF200 having the transcript ID (locus) NM_{—}003454 according to SEQUENCE LISTING SEQ. ID. NO. 10 and Table 1,
and combinations thereof.

42. The method according to claim 37, wherein the gene is selected from the group comprising:
STAT1 having the transcript ID (locus) NM_{—}007315 according to SEQUENCE LISTING SEQ. ID. NO. 11 and Table 2,
RTP4 having the transcript ID (locus) NM_{—}022147 according to SEQUENCE LISTING SEQ. ID. NO. 12 and Table 2,
UBE2L6 having the transcript ID (locus) NM_{—}004223 according to SEQUENCE LISTING SEQ. ID. NO. 13 and Table 2,
GBP1 having the transcript ID (locus) NM_{—}002053 according to SEQUENCE LISTING SEQ. ID. NO. 14 and Table 2,
CCL8 having the transcript ID (locus) NM_{—}005623 according to SEQUENCE LISTING SEQ. ID. NO. 15 and Table 2,
TNFSF10 having the transcript ID (locus) NM_{—}003810 according to SEQUENCE LISTING SEQ. ID. NO. 16 and Table 2,
and combinations thereof.

43. The method according to claim 37, wherein the depression is associated with the treatment and therapy of hepatitis.

44. The method according to claim 37, wherein the depression is associated with or caused by the administration of alpha-interferon in the treatment or therapy of hepatitis.

45. The method according to claim 37, wherein the depression is endogenous depression.

46. A method for finding and providing a diagnostic method for detecting depression or a medicament for the treatment of depression, wherein the method comprises using at least one nucleic acid molecule which induces depression or is associated with depression or at least one (poly)peptide encoded by the nucleic acid molecule, wherein the at least one nucleic acid molecule is selected from the group consisting of genes inducing depression or being associated with depression and respective DNA sequences thereof and related RNA sequences associated therewith.

47. The method according to claim 46, wherein the gene is selected from the group comprising:
DYNLT1 having the transcript ID (locus) NM_{—}006519 according to SEQUENCE LISTING SEQ. ID. NO. 1 and Table 1,
MEF2A having the transcript ID (locus) NM_{—}005587 according to SEQUENCE LISTING SEQ. ID. NO. 2 and Table 1,
TOR1B having the transcript ID (locus) NM_{—}014506 according to SEQUENCE LISTING SEQ. ID. NO. 3 and Table 1,
DISC1 having the transcript ID (locus) NM_{—}018662 according to SEQUENCE LISTING SEQ. ID. NO. 4 and Table 1,
GCH1 having the transcript ID (locus) NM_{—}000161 according to SEQUENCE LISTING SEQ. ID. NO. 5 and Table 1,
ST3GAL5 having the transcript ID (locus) NM_{—}003896 according to SEQUENCE LISTING SEQ. ID. NO. 6 and Table 1,
PSMB9 having the transcript ID (locus) NM_{—}002800 according to SEQUENCE LISTING SEQ. ID. NO. 7 and Table 1,
GLRX having the transcript ID (locus) NM_{—}002064 according to SEQUENCE LISTING SEQ. ID. NO. 8 and Table 1,
RBCK1 having the transcript ID (locus) NM_{—}006462 according to SEQUENCE LISTING SEQ. ID. NO. 9 and Table 1,
ZNF200 having the transcript ID (locus) NM_{—}003454 according to SEQUENCE LISTING SEQ. ID. NO, 10 and Table 1,
STAT1 having the transcript ID (locus) NM_{—}007315 according to SEQUENCE LISTING SEQ. ID. NO. 11 and Table 2,
RTP4 having the transcript ID (locus) NM_{—}022147 according to SEQUENCE LISTING SEQ. ID. NO. 12 and Table 2,
UBE2L6 having the transcript ID (locus) NM_{—}004223 according to SEQUENCE LISTING SEQ. ID. NO. 13 and Table 2,
GBP1 having the transcript ID (locus) NM_{—}002053 according to SEQUENCE LISTING SEQ. ID. NO. 14 and Table 2,
CCL8 having the transcript ID (locus) NM_{—}005623 according to SEQUENCE LISTING SEQ. ID. NO. 15 and Table 2,
TNFSF10 having the transcript ID (locus) NM_{—}003810 according to SEQUENCE LISTING SEQ. ID. NO. 16 and Table 2,
and combinations thereof.

48. The method according to claim 46, wherein the gene is selected from the group comprising:
DYNLT1 having the transcript ID (locus) NM_{—}006519 according to SEQUENCE LISTING SEQ. ID. NO. 1 and Table 1,
MEF2A having the transcript ID (locus) NM_{—}005587 according to SEQUENCE LISTING SEQ. ID. NO. 2 and Table 1,
TOR1B having the transcript ID (locus) NM_{—}014506 according to SEQUENCE LISTING SEQ. ID. NO. 3 and Table 1,
DISC1 having the transcript ID (locus) NM_{—}018662 according to SEQUENCE LISTING SEQ. ID. NO. 4 and Table 1,
GCH1 having the transcript ID (locus) NM_{—}000161 according to SEQUENCE LISTING SEQ. ID. NO. 5 and Table 1,
ST3GAL5 having the transcript ID (locus) NM_{—}003896 according to SEQUENCE LISTING SEQ. ID. NO. 6 and Table 1,
PSMB9 having the transcript ID (locus) NM_{—}002800 according to SEQUENCE LISTING SEQ. ID. NO. 7 and Table I,
GLRX having the transcript ID (locus) NM_{—}002064 according to SEQUENCE LISTING SEQ. ID. NO. 8 and Table 1,
RBCK1 having the transcript ID (locus) NM_{—}006462 according to SEQUENCE LISTING SEQ. ID. NO. 9 and Table 1, and
ZNF200 having the transcript ID (locus) NM_{—}003454 according to SEQUENCE LISTING SEQ. ID. NO. 10 and Table 1,
and combinations thereof.

49. The method according to claim 46, wherein the gene is selected from the group comprising:
STAT1 having the transcript ID (locus) NM_{—}007315 according to SEQUENCE LISTING SEQ. ID. NO. 11 and Table 2,
RTP4 having the transcript ID (locus) NM_{—}022147 according to SEQUENCE LISTING SEQ. ID. NO. 12 and Table 2,
UBE2L6 having the transcript ID (locus) NM_{—}004223 according to SEQUENCE LISTING SEQ. ID. NO. 13 and Table 2,
GBP1 having the transcript ID (locus) NM_{—}002053 according to SEQUENCE LISTING SEQ. ID. NO. 14 and Table 2,
CCL8 having the transcript ID (locus) NM_{—}005623 according to SEQUENCE LISTING SEQ. ID. NO. 15 and Table 2,
TNFSF10 having the transcript ID (locus) NM_{—}003810 according to SEQUENCE LISTING SEQ. ID. NO. 16 and Table 2,
and combinations thereof.

50. A method for identifying an inhibitor or repressor of a nucleic acid molecule which induces depression or is associated with depression, namely a gene or the DNA sequence thereof or the RNA sequence associated therewith, or of the corresponding (poly)peptide encoded by the nucleic acid molecule, the method comprising the following steps:
(a) Bringing the nucleic acid molecule or the corresponding (poly)peptide into contact with at least one test substance under conditions that allow interaction of the test substance(s) to the nucleic acid molecule or the corresponding (poly)peptide; and
(b) Detecting and analyzing whether the test substance(s) limit or prevent the gene activity or gene expression of the nucleic acid molecule or whether the test substance(s) limit or prevent the depression-inducing or depression-associated properties of the nucleic acid molecule or of the (poly)peptide.

51. The method according to claim 50, wherein multiple test substances are used and the following steps are carried out:
(a) Testing various test substances in different reaction vessels, whereby test substances which do not limit or prevent the depression-inducing or depression-associated properties of the nucleic acid molecule or of the (poly)peptide are not taken into account in the subsequent test method;
(b) Distributing test substances from such reaction vessels, in which reduction or prevention of depression-inducing or depression-associated properties of the nucleic acid molecule or of the (poly)peptide was determined in step (a), into new reaction vessels and repeating step (a) with the new reaction vessels; and
(c) Repeating step (b) until a single test substance is identified which may be associated with reduction or prevention of depression-inducing or depression-associated properties of the nucleic acid molecule or of the (poly)peptide.

52. The method according to one of claim 51, wherein the test substance(s), the nucleic acid molecule and the (poly)peptide are linked to a readout system and wherein the test assay is added to a readout system and wherein the readout system sends a detectable signal after the test substance(s) bind to the nucleic acid molecule or to the (poly)peptide.
